(19) United States
(12) Patent Application Publication
(10) Pub. No.: US 2005/0233446 A1
(43) Pub. Date: Oct. 20, 2005

(54) Defined media for stem cell culture

(76) Inventors: Xuejun Huang Parsons, San Diego, CA (US); Evan Y. Snyder, La Jolla, CA (US)

Correspondence Address: BIOTECHNOLOGY LAW GROUP, C/O PORTFOLIOIP, P.O. BOX 52050, MINNEAPOLIS, MN, 55402, US

(21) Appl. No.: 11/027,395

(22) Filed: Dec. 31, 2004

Related U.S. Application Data
(60) Provisional application No. 60/533,506, filed on Dec. 31, 2003.

(51) Int. Cl. C12N 5/08

(52) U.S. Cl. 435/366; 435/404

(57) ABSTRACT

Stem cells, including mammalian, and particularly primate primordial stem cells (pPSCs) such as human embryonic stem cells (hESCs), hold great promise for restoring cell, tissue, and organ function. However, cultivation of stem cells, particularly undifferentiated hESCs, in serum-free, feeder-free, and conditioned-medium-free conditions remains crucial for large-scale, uniform production of pluripotent cells for cell-based therapies, as well as for controlling conditions for efficiently directing their lineage-specific differentiation. This instant invention is based on the discovery of the formulation of minimal essential components necessary for maintaining the long-term growth of pPSCs, particularly undifferentiated hESCs. Basic fibroblast growth factor (bFGF), insulin, ascorbic acid, and laminin were identified to be both sufficient and necessary for maintaining hESCs in a healthy self-renewing undifferentiated state capable of both prolonged propagation and then directed differentiation. Having discerned these minimal molecular requirements, conditions that would permit the substitution of poorly-characterized and unspecified biological additives and substrates were derived and optimized with entirely defined constituents, providing a “biologics”-free (i.e., animal-, feeder-, serum-, and conditioned-medium-free) system for the efficient long-term cultivation of pPSCs, particularly pluripotent hESCs. Such culture systems allow the derivation and large-scale production of stem cells such as pPSCs, particularly pluripotent hESCs, in optimal yet well-defined biologics-free culture conditions from which they can be efficiently directed towards a lineage-specific differentiated fate in vitro, and thus are important, for instance, in connection with clinical applications based on stem cell therapy and in drug discovery processes.

### INCORPORATION BY REFERENCE

This application claims benefit of and priority to U.S. provisional patent application Ser. No. 60/533,506, filed 31 Dec. 2003, which is hereby incorporated by reference as if fully set forth.

### GOVERNMENT INTEREST

This invention was made with government support under NS040822 awarded by the National Institutes of Health. The government has certain rights in this invention.

### TECHNICAL FIELD

The present invention relates to cell culture technology. Specifically, the invention concerns serum-free defined media that can be used for the long-term cultivation of primordial stems cells from primates in a substantially undifferentiated state.

### BACKGROUND OF THE INVENTION

1. Introduction

The following description includes information that may be useful in understanding the present invention. It is not an admission that any such information is prior art, or relevant, to the presently claimed inventions, or that any publication specifically or implicitly referenced is prior art.

2. Background

Stem cells are cells capable of differentiation into other cell types, including those having a particular, specialized function (i.e., terminally differentiated cells, such as erythrocytes, macrophages, etc.), progenitor (i.e., “multipotent”) cells which can give rise to any one of several different terminally differentiated cell types, and cells that are capable of giving rise to various progenitor cells. Cells that give rise to some or many, but not all, of the cell types of an organism are often termed “pluripotent” stem cells, which are able to differentiate into any cell type in the body of a mature organism, although without reprogramming they are unable to de-differentiate into the cells from which they were derived. As will be appreciated, “multipotent” stem/progenitor cells (e.g., neural stem cells) have a more narrow differentiation potential than do pluripotent stem cells. Another class of cells even more primitive (i.e., uncommitted to a particular differentiation fate) than pluripotent stem cells are the so-called “totipotent” stem cells (e.g., fertilized oocytes, cells of embryos at the two and four cell stages of development), which have the ability to differentiate into any type of cell of the particular species. For example, a single totipotent stem cell could give rise to a complete animal, as well as to any of the myriad of cell types found in the particular species (e.g., humans). In this specification, pluripotent and totipotent cells, as well as cells with the potential for differentiation into a complete organ or tissue, are referred as “primordial” stem cells.

As can be appreciated, there is great interest in isolating and growing stem cells, especially primordial stem cells, from different species, particularly from primates, and especially from humans, since such primordial stem cells could provide a supply of readily available cells and tissues of all types for use in transplantation, cell regeneration and replacement therapy, drug discovery, generation of model systems for studying mammalian development, and gene therapy.

Standing in the way of this result, however, is the reality that to date only several sub-optimal methods for isolating and growing primordial stem cells from primates have been reported. Unfortunately, these methods are not as straightforward as, and are relatively inefficient compared with, methods for culturing primordial stem cells for other non-primate species such as mouse. For example, murine embryonic stem cells can be maintained in an undifferentiated state using feeder-free cultures that have been supplemented with leukemia inhibitory factor (LIF). On the other hand, conventional techniques for maintaining human embryonic stem cells lead to their rapid differentiation when the cells are cultured without an appropriate feeder cell layer or conditioned medium from a suitable feeder cell line, even in the presence of LIF.

Additionally, current methods of culturing undifferentiated primate primordial stem cells require such things as the use of serum in addition to a feeder cell layer (or conditioned medium from an appropriate feeder cell line). Moreover, systems that employ feeder cells (or conditioned media from feeder cell cultures) often use cells from a different species than that of the stem cells being cultivated. For instance, growth-arrested mouse embryonic fibroblasts (MEF) have traditionally been used as the feeder layer to maintain a long-term undifferentiated growth of human embryonic stem cells. Though there has been a report of a feeder-free system for cultivating human embryonic stem cells, it requires the use of conditioned medium from MEF cultures in order to maintain the stem cells in an undifferentiated state.

The requirement for components such as serum, feeder cells, and/or conditioned medium complicates the process of cultivating primate primordial stem cells. Moreover, the use of cells, especially xenogeneic cells (or cell products), increases the risk that the resulting primordial stem cell populations produced by such methods may be contaminated with unwanted components (e.g., aberrant cells, viruses, cells that may induce an immune response in a recipient of the stem cell population, heterogeneous fusion cells, etc.), thereby comprising, for example, the therapeutic potential of human embryonic stem cells cultured by such methods. To address the limitations imposed by using xenogeneic feeder cells or conditioned medium from xeno cultures, techniques have recently been developed for culturing human embryonic stem cells that use feeder cell layers made from human fetal and adult fibroblasts, human foreskin fibroblasts, and human adult marrow stromal cells. However, like other conventional human embryonic stem cells culturing techniques, those that use human feeder cells still suffer from the drawback of exposing the undifferentiated cells to undefined culture conditions, serum, and/or conditioned medium. As such, the conditions cannot be optimized, and unwanted differentiation-inducing, pathogenic, or toxic factors may be present.

Clearly, the formulation of an optimal culture media for propagating undifferentiated primate primordial stem cells would be beneficial, and would allow for large-scale, uniform production of undifferentiated primate primordial stem cells, as well as lineage-specific cells derived therefrom by subsequent manipulation. Access to large, well-defined supplies of such cells is crucial to their use in cell-based therapies and for other purposes.

3. Definitions

When used in this specification, the following terms will be defined as provided below unless otherwise stated. All other terminology used herein will be defined with respect to its usage in the particular art to which it pertains unless otherwise noted.

“Basal medium” refers to a solution of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cells' survival. These are compounds that the cells themselves can not synthesize, due to the absence of one or more of the gene(s) that encode the protein(s) necessary to synthesize the compound (e.g., essential amino acids) or, with respect to compounds which the cells can synthesize, because of their particular developmental state the gene(s) encoding the necessary biosynthetic proteins are not being expressed as sufficient levels. A number of base media are known in the art of mammalian cell culture, such as Dulbecco's Modified Eagle Media (DMEM), Knockout-DMEM (KO-DMEM), and DMEM/F12, although any base medium that can be supplemented with bFGF, insulin, and ascorbic acid and which supports the growth of primate primordial stem cells in a substantially undifferentiated state can be employed.

“Conditioned medium” refers to a growth medium that is further supplemented with soluble factors derived from cells cultured in the medium. Techniques for isolating conditioned medium from a cell culture are well known in the art. As will be appreciated, conditioned medium is preferably essentially cell-free. In this context, “essentially cell-free” refers to a conditioned medium that contains fewer than about 10%, preferably fewer than about 5%, 1%, 0.1%, 0.01%, 0.001%, and 0.0001% than the number of cells per unit volume, as compared to the culture from which it was separated.

A “defined” medium refers to a biochemically defined formulation comprised solely of the biochemically-defined constituents. A defined medium may include solely constituents having known chemical compositions. A defined medium may also include constituents that are derived from known sources. For example, a defined medium may also include factors and other compositions secreted from known tissues or cells; however, the defined medium will not include the conditioned medium from a culture of such cells. Thus, a “defined medium” may, if indicated, include a particular compounds added to form the culture medium, up to and including a portion of a conditioned medium that has been fractionated to remove at least one component detectable in a sample of the conditioned medium that has not been fractionated. Here, to “substantially remove” of one or more detectable components of a conditioned medium refers to the removal of at least an amount of the detectable, known component(s) from the conditioned medium so as to result in a fractionated conditioned medium that differs from an unfractionated conditioned medium in its ability to support the long-term substantially undifferentiated culture of primate stem cells. Fractionation of a conditioned medium can be performed by any method (or combination of methods) suitable to remove the detectable component(s), for example, gel filtration chromatography, affinity chromatography, immune precipitation, etc. Similarly, or a “defined medium” may include serum components derived from an animal, including human serum components. In this context, “known” refers to the knowledge of one of ordinary skill in the art with reference to the chemical composition or constituent.

“Embryonic germ cells” or “EG cells” are cells derived from the primordial germ cells of an embryo or fetus that are destined to give rise to sperm or eggs. EG cells are among the embryonic stem cells that can be cultured in accordance with the invention.

“Embryonic stem cells” or “ES cells” are cells obtained from an animal (e.g., a primate, such as a human) embryo, preferably from an embryo that is less than about eight weeks old. Preferred embryonic stages for isolating primordial embryonic stem cells include the morula or blastocyst stage of a pre-implantation stage embryo.

“Extracellular matrix” or “matrix” refers to one or more substances that provide substantially the same conditions for supporting cell growth as provided by an extracellular matrix synthesized by feeder cells. The matrix may be provided on a substrate. Alternatively, the component(s) comprising the matrix may be provided in solution.

“Feeder cells” are non-primordial stem cells on which stem cells, particularly primate primordial stem cells, may be plated and which provide a milieu conducive to the growth of the stem cells.

A cell culture is “essentially feeder-free” when it does not contain exogenously added conditioned medium taken from a culture of feeder cells nor exogenously added feeder cells in the culture, where “no exogenously added feeder cells” means that cells to develop a feeder cell layer have not been purposely introduced for that reason. Of course, if the cells to be cultured are derived from a seed culture that contained feeder cells, the incidental co-isolation and subsequent introduction into another culture of some small proportion of those feeder cells along with the desired cells (e.g., undifferentiated primate primordial stem cells) should not be deemed as an intentional introduction of feeder cells. Similarly, feeder cells or feeder-like cells that develop from stem cells seeded into the culture shall not be deemed to have been purposely introduced into the culture.

A “growth environment” is an environment in which stem cells (e.g., primate primordial stem cells) will proliferate in vitro. Features of the environment include the medium in which the cells are cultured, and a supporting structure (such as a substrate on a solid surface) if present.

“Growth factor” refers to a substance that is effective to promote the growth of stem cells and which, unless added to the culture medium as a supplement, is not otherwise a component of the basal medium. Put another way, a growth factor is a molecule that is not secreted by cells being cultured (including any feeder cells, if present) or, if secreted by cells in the culture medium, is not secreted in an amount sufficient to achieve the result obtained by adding the growth factor exogenously. Growth factors include, but are not limited to, basic fibroblast growth factor (bFGF), acidic fibroblast growth factor (aFGF), epidermal growth factor (EGF), insulin-like growth factor-I (IGF-I), insulin-like growth factor-II (IGF-II), platelet-derived growth factor-AB (PDGF), and vascular endothelial cell growth factor (VEGF), activin-A, and bone morphogenic proteins (BMPs), insulin, cytokines, chemokines, morphogents, neutralizing antibodies, other proteins, and small molecules.

“Isotonic” refers to a solution having essentially the same tonicity (i.e., effective osmotic pressure equivalent) as another solution with which it is compared. In the context of cell culture, an “isotonic” medium is one in which cells can be cultured without an appreciable net flow of water across the cell membranes.

A solution having “low osmotic pressure” refers to a solution having an osmotic pressure of less than about 300 milli-osmols per kilogram (“mOsm/kg”).

A “normal” stem cell refers to a stem cell (or its progeny) that does not exhibit an aberrant phenotype or have an aberrant genotype, and thus can give rise to the full range of cells that be derived from such a stem cell. In the context of a totipotent stem cell, for example, the cell could give rise to, for example, an entire, normal animal that is healthy. In contrast, an “abnormal” stem cell refers to a stem cell that is not normal, due, for example, to one or more mutations or genetic modifications or pathogens. Thus, abnormal stem cells differ from normal stem cells.

A “non-essential amino acid” refers to an amino acid species that need not be added to a culture medium for a given cell type, typically because the cell synthesizes, or is capable of synthesizing, the particular amino acid species. While differing from species to species, non-essential amino acids are known to include L-alanine, L-asparagine, L-aspartic acid, L-glutamic acid, glycine, L-proline, and L-serine.

A “primate-derived primordial stem cell” or “primate primordial stem cell” is a primordial stem cell obtained from a primate species, including humans and monkeys, and includes genetically modified primordial stem cells.

“Pluripotent” refers to cells that are capable of differentiating into one of a plurality of different cell types, although not necessarily all cell types. An exemplary class of pluripotent cells is embryonic stem cells, which are capable of differentiating into any cell type in the human body. Thus, it will be recognized that while pluripotent cells can differentiate into multipotent cells and other more differentiated cell types, the process of reverse differentiation (i.e., de-differentiation) is likely more complicated and requires “re-programming” the cell to become more primitive, meaning that, after re-programming, it has the capacity to differentiate into more or different cell types than was possible prior to re-programming.

A cell culture is “essentially serum-free” when it does not contain exogenously added serum, where no “exogenously added feeder cells” means that serum has not been purposely introduced into the medium. Of course, if the cells being cultured produce some or all of the components of serum, of if the cells to be cultured are derived from a seed culture grown in a medium that contained serum, the incidental co-isolation and subsequent introduction into another culture of some small amount of serum (e.g., less than about 1%) should not be deemed as an intentional introduction of serum.

“Substantially undifferentiated” means that population of stem cells (e.g., primate primordial stem cells) contains at least about 50%, preferably at least about 60%, 70%, or 80%, and even more preferably, at least about 90%, undifferentiated, stem cells. Fluorescence-activated cell sorting using labeled antibodies or reporter genes/proteins (e.g., enhanced green fluorescence protein [EGFP]) to one or more markers indicative of a desired undifferentiated state (e.g., a primordial state) can be used to determine how many cells of a given stem cell population are undifferentiated. For purposes of making this assessment, one or more of cell surface markers correlated with an undifferentiated state (e.g., Oct-4, SSEA-4, Tra-1-60, and Tra-1-81) can be detected. Telomerase reverse transcriptase (TERT) activity and alkaline phosphatase can also be assayed. In the context of primate primordial stem cells, positive and/or negative selection can be used to detect, for example, by immuno-staining or employing a reporter gene (e.g., EGFP), the expression (or lack thereof) of certain markers (e.g., Oct-4, SSEA-4, Tra-1-60, Tra-1-81, SSEA-1, SSEA-3, nestin, telomerase, Myc, p300, and Tip60 histone acetyltransferases, and alkaline phosphatase activity) or the presence of certain post-translational modifications (e.g., acetylated histones), thereby facilitating assessment of the state of self-renewal or differentiation of the cells.

“Totipotent” refers to cells that are capable of differentiating into any cell type, including pluripotent, multipotent, and fully differentiated cells (i.e., cells no longer capable of differentiation into various cell types), such as, without limitation, embryonic stem cells, neural stem cells, bone marrow stem cells, hematopoietic stem cells, cardiomyocytes, neuron, astrocytes, muscle cells, and connective tissue cells.

### SUMMARY OF THE INVENTION

The object of this invention is to provide defined media that supports the long-term cultivation of stem cells, including undifferentiated primate stem cells, particularly primate primordial stem cells (e.g., human embryonic stem cells). The media is essentially free of serum, and feeder cells or feeder cell-conditioned medium is not required.

Thus, in one aspect, the invention concerns defined media useful in culturing stem cells, including undifferentiated primate primordial stem cells. In solution, the media is substantially isotonic as compared to the stem cells being cultured. In a given culture, the particular medium comprises a base medium and an amount of each of bFGF, insulin, and ascorbic acid necessary to support substantially undifferentiated growth of the primordial stem cells. In preferred embodiments, the base medium comprises salts, essential amino acids, and a carbon source that can be metabolized by primate stem cells, all in an amount that will support substantially undifferentiated growth of primate stem cells. Particularly preferred is a base medium of DMEM, or KO-DMEM, or DMEM/F12 that comprises essential amino acids and glucose. Preferably, the medium has a low endotoxin level. A medium according to the invention can also be supplemented with any compound(s) that will not interfere with, and preferably supports the maintenance of, culturing the stem cells in an undifferentiated state over time. Preferred examples of such compounds include non-essential amino acids, anti-oxidants, reducing agents, vitamins, organic compounds, inorganic salts, transferring, and albumins.

The invention's culture media also each comprise bFGF, insulin, and ascorbic acid. Preferably, the amount of bFGF will range from about 1 ng/mL (nanogram/mL) to about 50 μg/mL (microgram/mL) of culture. A concentration of about 20 ng/mL bFGF is currently particularly preferred. The amount of insulin can also be varied, preferably within the range of about 1 ng/mL to about 20 mg/mL, with a concentration of about 20 jig/mL being particularly preferred. Ascorbic acid concentrations can also vary, preferably over the range of from about 1 ng/mL to about 50 mg/mL, with about 50 μg/mL being particularly preferred.

Preferred cell types that can be cultured in an undifferentiated state using the media of the invention include stem cells derived from humans, monkeys, and apes. With regard to human stem cells, human primordial stem cells are preferred, particularly those derived from an embryo, preferably from a pre-implantation embryo, such as from a blastula or a morula.

Closely related aspects concern systems and methods for culturing stem cells such as primordial primate stem cells in a substantially undifferentiated state using a defined medium according to the invention. With regard to such systems, they comprise a culture medium according to the invention and a cell culture vessel that typically includes a substrate comprising a matrix that supports the undifferentiated growth of primate primordial stem cells. In certain preferred embodiments, the substrate is a solid, such as a plastic, ceramic, metal, or other biocompatible material to which cells can adhere, or to which a composition (e.g., a matrix) to which cells can adhere can be attached. In other embodiments, the matrix component(s) are in solution so as to facilitate suspension culture. The culture vessel can be as small as a well in multi-well tissue culture plate, or as large as a large stirred tank bioreactor. For preferred large-scale applications, to increase the available surface area for cell attachment, any suitable microcarrier (e.g., plastic beads or polymers) or the like may be used. In such cases, the microcarriers serve as the substrate. Any suitable matrix is attached to the substrate. The matrix can be made of cells, for example, it can be comprised of a primate feeder cell layer, wherein the cells are preferably of the same species (i.e., are allogeneic) as the primate stem cells being cultured. In embodiments where human stem cells are to be cultured, preferred cell-based matrices include those comprised of human fibroblast or stromal cells. Alternatively, the matrix can be substantially cell-free and is typically comprised of one or more extracellular matrix components, e.g., laminin, fibronectin, collagen, and gelatin, preferably laminin or combination of matrix components that contain laminin or other components that induce signaling pathways that enable the stem cells to continue to grow in a substantially undifferentiated state.

Because the culture systems of the invention are useful for the long-term maintenance of stem cells such as undifferentiated primate primordial stem cells, they typically comprise a plurality of culture vessels such that an aliquot containing dissociated stem cell colonies and/or dissociated single stem cells from one culture can be passaged to another vessel (preferably of the same sort) for continued culturing in a substantially undifferentiated state.

The culture methods of the invention comprise culturing stem cells such as primate primordial stem cells in a growth environment that is essentially feeder-free and serum-free and which comprises a defined, isotonic culture medium according to the invention and a matrix (for example, but not restricted to, laminin) attached to a substrate or in solution. Such defined, isotonic culture media contain the essential components that are required for maintaining the stem cells (e.g., primate primordial stem cells) in a substantially undifferentiated state, e.g., bFGF, insulin, and ascorbic acid (or their functional equivalents). The cells can be cultured in such an environment in any suitable culture vessel under conditions that allow an undifferentiated state to be maintained.

Using such methods, populations of stem cells, including substantially undifferentiated primate primordial stem cells, e.g., human embryonic stem cells, can be isolated from the resulting cell cultures, thereby representing another aspect of the invention. Such populations can be isolated by any suitable technique. Such techniques include affinity chromatography, panning, and fluorescence-assisted cell sorting. Such techniques each employ one or more separation reagents (for example, but not restricted to, antibodies and antibody fragments, reporter genes/proteins, etc.) that are specific for a cell-based marker indicative of an undifferentiated state. In the context of substantially undifferentiated human embryonic stem cells, such markers include, for example, but not restricted to the transcriptional factor Oct-4, and cell surface markers SSEA-4, Tra-1-60, and Tra-1-81. Other markers include telomerase, Myc, p300, and Tip60 histone acetyltransferases, acetylated histones, and alkaline phosphatase. Negative selection can also be employed, whereby cells that express one or more markers indicative of other than a substantially undifferentiated state, or alternatively, cells which fail to express a particular marker, can be removed from the desired cell population. Such populations can be used to produce stable stem cell lines, including cell lines of primate primordial stem cells such as human embryonic stem cells. If desired, such cells can be genetically modified to, for example, alter (i.e., increase or decrease) the expression of one or more endogenous genes, and/or express one or more genes introduced into the cells. Such genetic modifications can serve, for example, to correct genetic defects detected in a particular stem cell line, as well as to generate abnormal cell lines (which may be useful as model systems that mimic or replicate a genetic context correlated with a particular disease state).

Yet other aspects of the invention relate to methods of using stem cells, including substantially undifferentiated primate primordial stem cells, cultured or isolated in accordance with the invention. For instance, such cells can be used to identify factors that promote the cells' differentiation, or, alternatively, their continued maintenance in a substantially undifferentiated state or de-differentiation to a more primitive state (e.g., going from a multipotent stem cell to a pluripotent or totipotent stem cell). Briefly, in the context of differentiation or maintenance of a substantially undifferentiated state, such methods involve, for example, exposing a test compound to substantially undifferentiated primate primordial stem cells that are being cultured in a defined, isotonic culture medium of the invention. Following exposure to the test compound, the cells are assessed to determine if they have been better maintained in a substantially undifferentiated state or induced to differentiate. If the cells have been better maintained in a substantially undifferentiated state, the test compound can be identified as one that promotes an undifferentiated state or self-renewal of primate primordial stem cells. If the cells have been induced to differentiate, the test compound can be identified as one that promotes differentiation of substantially undifferentiated primate primordial stem cells. The differentiating cells may be followed to determine their developmental fate, in other words, to determine what cell lineage they become as a result of differentiating. In the context of de-differentiation, cells of a more differentiated state (e.g., hematopoietic stem cells) are exposed to one or more compounds and then assessed to determine if the exposure resulted in cells of a more primitive type (e.g., a primordial stem cell) than those initially exposed to the test compound. If so, the compound that produces the effect is identified as one that promotes de-differentiation, or reprogramming, of cells. Preferably, these and other screening methods according to the invention are conducted in a high throughput manner, such that numerous compounds can be simultaneously screened.

Another aspect of the invention comprises isolation, establishment, and culturing of stem cell lines, including primate primordial stem cell lines, particularly undifferentiated human embryonic stem cell lines, in an allogeneic, defined growth environment according to the invention. For example, primate primordial stem cells cultured in accordance with the invention, particularly pluripotent undifferentiated human embryonic stem cells (hESCs) and their derivatives (e.g., hESC-derived multipotent neural stem cells, hematopoietic precursor cells, cardiomyocytes, and insulin-producing cells) that are cultivated and maintained in a xeno-free growth environment, can be used therapeutically. Representative therapeutic uses include cell-based therapies to treat disorders such as heart diseases, diabetes, liver diseases, neurodegenerative diseases, cancers, tumors, strokes, spinal cord injury or diseases, Alzheimer's diseases, Parkinson's diseases, multiple sclerosis, amyotrophic lateral sclerosis (ALS), and disorders caused by single gene defects. In such methods, a patient in need of such therapy is administered a population of substantially undifferentiated human embryonic stem cells or differentiated cells derived from substantially undifferentiated human embryonic stem cells. The cells so administered may be genetically modified, although this is not essential.

Another aspect of the invention concerns methods of directing the fate, in terms of differentiation toward a specific tissue or cell lineage, of stem cells, particularly primate primordial stem cells. In preferred examples of such methods, substantially undifferentiated primate primordial stem cells (e.g., human embryonic stem cells), for instance, are induced to differentiate into a particular cell type or lineage by administering one or more factors that promote such differentiation. Conversely, the invention also concerns methods for re-programming more developmentally committed cells to become more primitive or immature. For instance, human hematopoietic stem cells are induced to de-differentiate into cells that can give rise to cell types not only of the hematopoietic lineage but also other, non-hematopoietic cell types.

Other features and advantages of the invention will be apparent from the following brief description of the figures, detailed description, and appended claims.

### DESCRIPTION OF THE FIGURES

FIGS. 1-6 represent data from the experiments described in the Example section, below.

FIG. 1: Basic fibroblast growth factor (bFGF) is a critical component in a defined hESC medium that sustains undifferentiated growth of human embryonic stem cells (hESCs).

(a) Characterization of hESCs maintained on growth-arrested human foreskin fibroblast (HFF) cells and laminin/collagen-coated plates. Phase images [phase] show the highly compact undifferentiated morphology of an hESC colony grown on human feeder cells [A] or on plates coated with the commercially available combination of laminin and collagen (known as Matrigel) [K]. White arrows delineate the edge of an hESC colony. Red stars in [A] indicate the large human foreskin fibroblasts (HFF) that compose the feeder layer. Red arrows in [A, B] and [K, L] indicate the elliptoid-appearing differentiated hESCs that have migrated beyond the colony. The area delineated by the white square in [A] indicates the approximate area that is visualized at higher magnification in [B-J] and in [L-T]. Immunofluorescence analysis indicates that hESCs inside the colonies maintained on human feeder cells and Matrigel-coated plates express the undifferentiated hESC markers Oct-4 [C, D, M, N] (red), SSEA-4 [E, F, O, P] (red), Tra-1-60 [G, H, Q, R] (red), and Tra-1-81 [I, J, S, T] (red). Cells at the edge of the colonies exhibit the classic flattened epithelial morphology indicative of the onset of differentiation, and express the stem cell surface marker most suggestive of imminent differentiation, SSEA-3 [B, L] (red) and nestin, an intermediate filament associated with cells of early neuroectoderm [B, L] (green). Cells that have migrated outside the colonies have continued to differentiate into large elliptoid-appearing cells that persist in expressing nestin, but cease expressing SSEA-3, Oct-4, SSEA-4, Tra-1-60, and Tra-1-81 [B-J, L-T]. Note that the colonies on laminin/collagen have a more uniform morphology than those grown on HFFs, as indicated by the presence of a narrower edge of SSEA-3 positive cells ([L] compared to [B]). All cells in [B-J] and [L-T] are revealed by DAPI staining of their nuclei (blue). [D], [F], [H], [J], [N], [P], [R], and [T] are the images in [C], [E], [G], [I], [M], [O], [Q], and [S], respectively, merged with DAPI staining of their nuclei (blue).

(b) Short-term proliferation assays—assessing cell number. The growth rate of hESCs maintained under the feeder-free condition in the defined hESC media containing 0, 4, 10, 20, 30, or 50 ng/ml bFGF were determined and compared to that of hESCs maintained on laminin/collagen-coated plates in the MEF-conditioned media (MEF-CM) containing 10 ng/ml bFGF (see, for example, Xu, C., et al., Nat. Biotechnol. 19, 971-974 (2001)). In the defined media containing no bFGF or a low concentration of bFGF (4 ng/ml), hESCs displayed significantly slow growth rates. In hESC media supplemented with bFGF at a concentration ranging from 10 to 50 ng/ml, hESCs displayed a comparable growth rate as those maintained in MEF-CM, suggesting that bFGF is a critical growth factor for hESC propagation and may substitute for MEF-conditioned media.

(c) bFGF dose-response assays—assessing maintenance of the undifferentiated state. The percentage of undifferentiated hESCs after 7 days of culturing under the feeder-free condition in the defined hESC media containing 0, 4, 10, 20, 30, or 50 ng/ml bFGF were determined and compared to that of hESCs maintained on laminin/collagen-coated plates in the MEF-CM containing 10 ng/ml bFGF (see, for example, Xu, C., et al., (2001)) In the defined media containing no bFGF or a low concentration of bFGF (4 ng/ml), hESCs displayed high percentages of differentiation. While the percentage of undifferentiated hESC colonies increased with the increased bFGF concentration (up to 20 ng/ml), slightly decreased percentages of undifferentiated hESC colonies were observed with higher dosages of bFGF (30 and 50 ng/ml). hESCs maintained in media containing 20 ng/ml bFGF exhibited the highest percentage of undifferentiated hESC colonies that is comparable to those sustained in MEF-CM, further suggesting that bFGF is the critical component in the defined hESC media that sustains undifferentiated growth. In other words, taken together with the graph in (b), these data suggest that 20 ng/ml bFGF provides the greatest number of undifferentiated cells and, at a level comparable to MEF-conditioned media, may substitute for this undefined component.

(d) bFGF is critical for sustaining undifferentiated growth of hESCs carrying an Oct-4-driven reporter gene. hESCs carrying a reporter gene (enhanced green fluorescence protein [EGFP]) that is under control of the Oct-4 promoter was generated via lentiviral-mediated transduction. Transfected hESC colonies cultivated under the feeder-free conditions displayed undifferentiated morphology and a strong green fluorescence (Oct-4 expression) in the defined media containing 20 ng/ml bFGF [A, B], comparable to those maintained in MEF-CM [E, F], while over 70% of cells inside the colonies displayed a differentiated morphology and ceased Oct-4 expression in the absence of bFGF upon first passage (day 7 after seeding) [C, D].

(e) bFGF is essential for maintaining hESCs in a healthy undifferentiated state, in part through MAPK signaling deactivation. In media lacking bFGF, hESC colonies maintained on Matrigel-coated plates have a completely differentiated morphology upon the first passage [A]. To examine the signaling pathways that might be mediated by bFGF, the phosphorylation level of p38 MAPK in undifferentiated (in the presence of bFGF [+bFGF]) and differentiated (in the absence of bFGF [−bFGF]) hESCs was examined. An unphosphorylated inactive form of p38 (green cells) was observed in undifferentiated hESCs maintained in the defined media containing 20 ng/ml bFGF [B]. Although, in the absence of bFGF, the unphosphorylated form of p38 remained present in most of the large cells inside the differentiated hESC colony, a subpopulation (˜5%) of the large differentiated cells displayed high level of p38 phosphorylation [“p-p38”, red cells, C], suggesting that the p38 MAPK signaling was activated and might be involved in differentiation of those cells. White arrows delineate the edge of an hESC colony.

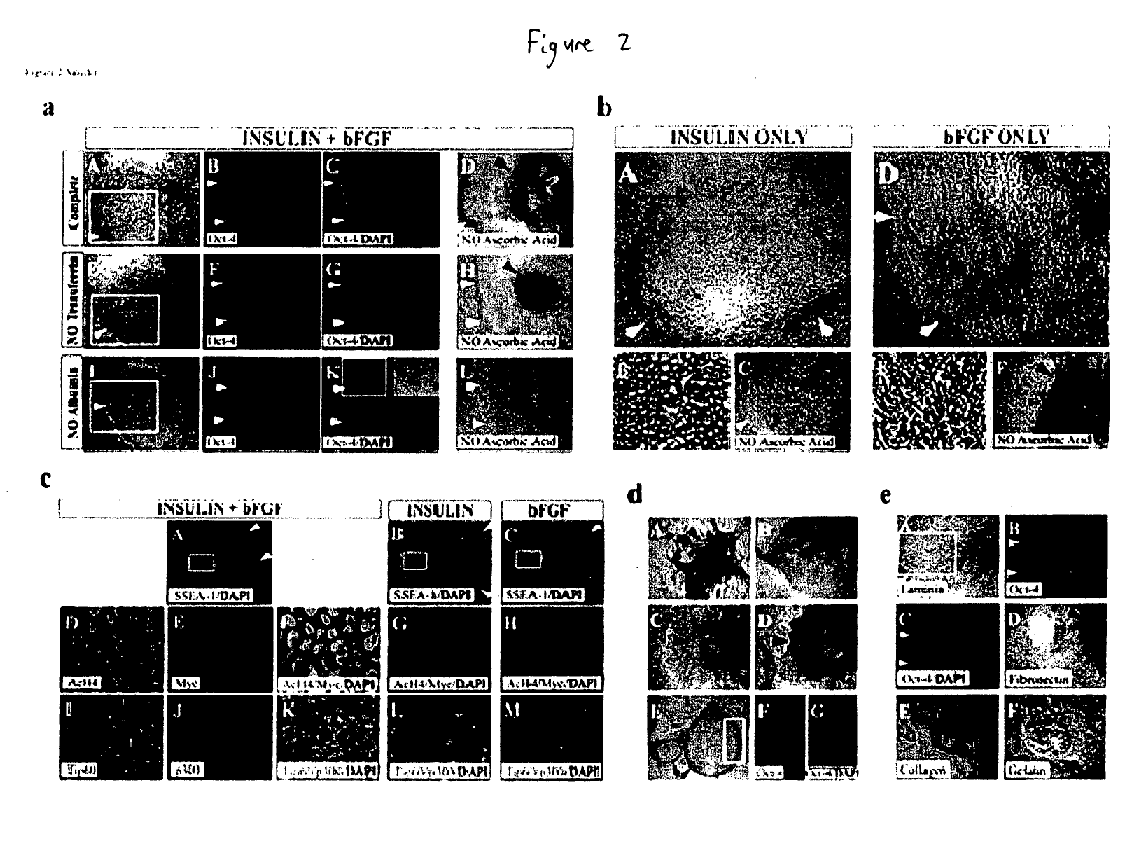

FIG. 2: Basic fibroblast growth factor (bFGF), insulin, ascorbic acid, and laminin (a “biologics”-free formulation) are minimal essential requirements for growth of undifferentiated hESCs on a matrix.

(a) Media containing bFGF, insulin, and ascorbic acid sustain the healthy undifferentiated growth of hESCs on laminin/collagen-coated plates. Insulin (20 μg/ml), transferrin (8 μg/ml), albumin (for example, the commercial product known as AlbuMAXI) (10 mg/ml), and ascorbic acid (50 μg/ml) were added to a base medium that consisted of 100% DMEM/F-12 with 20 ng/ml bFGF, 2 mM L-alanyl-L-glutamine, 1× MEM essential amino acids solution, 1× MEM non-essential amino acids solution, and 100 μM β-mercaptoethanol. The degree of differentiation was judged by morphology of the colonies and Oct-4 expression. When all of the components were present [A-C], the majority of hESC colonies displayed a highly compact undifferentiated morphology [A] and expressed Oct-4 [B, C] (red), indicating that these factors were sufficient to support undifferentiated growth of hESC s. In the absence of transferrin [E-G], fewer total hESC colonies were observed; however, the hESC colonies that were observed had a high proportion with a highly compact undifferentiated morphology [E] and that expressed Oct-4 [F, G] (red). In the absence of AlbuMax [I-K], hESC colonies were more flat and spread out (as seen in the DIC image in the inset in [K]; the white square delineates the same area shown in the inset), but a high proportion of the cells continued to express Oct-4 [J, K] (red) and exhibited a highly compact undifferentiated morphology [I]. However, if ascorbic acid was omitted from the media [D, H, L] (“NO Ascorbic Acid”), the colonies often became very dense in the center and necrotic [D, H, L, red arrows], indicating that ascorbic acid is likely essential for maintaining healthy undifferentiated growth. White arrows in all panels delineate the edge of an hESC colony. The area delineated by the white square in [A], [E], and [I] indicates the approximate area that is visualized at higher magnification in [B, C], [F, G], and [J, K], respectively. [C], [G], and [K] is the image in [B], [F], and [J], respectively, merged with DAPI staining of their nuclei (blue).

(b) When either bFGF or insulin was omitted from the media, hESC colonies appeared to differentiate completely under the feeder- and serum-free conditions. Usually, large round cells were present in media that contained only insulin [A, B] (phase), and elliptically-shaped cells were present in media that contained only bFGF [D, E] (phase), suggesting that insulin and bFGF might have distinct effects on the fate of hESC s. The distinct growth effects of insulin and bFGF were further accentuated in media lacking ascorbic acid [C, F] (“NO Ascorbic Acid”). In the absence of ascorbic acid and in media that contained only insulin as the growth factor [C] (phase) (“NO Ascorbic Acid”), slower growth of the differentiated hESC s was observed (compare [A, B] to [C]). In ascorbic-acid-lacking media that contained bFGF as the only growth factor [F] (phase) (“NO Ascorbic Acid”), the presence of dense centers with cyst-like structures and necrotic cells within the differentiated regions of growing hESC colonies became more severe (compare [D, E] to [F], red arrows), suggesting that the combination of bFGF, insulin and ascorbic acid are all essential for maintaining the health, well-being, and continued propagation of hESC s in an undifferentiated state. White arrows in all panels delineate the edge of hESC colonies.

(c) Presence of both bFGF and insulin is essential for maintenance of an acetylated transcriptionally active chromatin state in undifferentiated hESCs. When either bFGF or insulin is omitted from the media, the differentiated hESC colonies express the differentiation-associated cell surface marker SSEA-1 [B, C] (red); while undifferentiated hESCs maintained in media containing both bFGF and insulin appropriately do not express SSEA-1 [A]. In the presence of both bFGF and insulin, undifferentiated hESCs displayed strong immunoreactivity to acetylated histone H4 [D, F] (green), Myc [E, F] (red), and histone acetyltransferase (HAT) Tip6O [I, K] (green) and p300 [J, K] (red), suggesting an acetylated transcriptionally active chromatin state. However, when either bFGF or insulin was omitted from the media, the differentiated cells showed undetectable or weak immunoreactivity to acetylated H4, Myc [G, H], Tip60 HAT, and nuclear focal localization of p300 HAT [L, M], suggesting an hypoacetylated repressed chromatin state. All cells are indicated by DAPI staining of their nuclei (blue).

(d) The following human growth factors—aFGF, EGF, IGF-I, IGF-II, PDGF, VEGF, activin-A, and BMP-2—can not replace bFGF in supporting undifferentiated growth of hESCs under the feeder- and serum-free conditions. Although colony morphologies differ slightly depending on the growth factor used (representative colonies are shown in [A-D]), hESC colonies maintained in these above growth factors generally display a more differentiated morphology that consists of dense centers containing cyst-like structures and necrotic cells [red arrows] surrounded by a flat layer of fibroblast-like cells, suggesting that none of these factors can replace bFGF in maintaining undifferentiated healthy growth of hESCs. Note that, although most cells are differentiated, a minority of the small colonies (<30%) retain a compact morphology [E, blue arrows] and continue to express Oct-4 [F, G] (red). The area delineated by the white square in [E] indicates the approximate area that is visualized at higher magnification in [F, G]. [G] is the image in [F] merged with DAPI staining of their nuclei (blue). (Although some cells in the center of the colony in [A-D] appear to be pigmented, this is actually an optical illusion created by the dense necrotic cells heaping upon each other.)

(e) Characterization of hESCs maintained on matrix protein-coated plates in the defined hESC media—Determining the Minimal Essential Matrix. hESCs maintained on a laminin-coated plate have a classic undifferentiated morphology [A] (phase image) and express Oct-4 [B, C] (red). [C] is the image in [B] merged with DAPI staining of their nuclei (blue). White arrows delineate the edge of an hESC colony. The area delineated by the white square in [A] indicates the approximate area that is visualized at higher magnification in [B, C]. In contrast, hESC colonies maintained on fibronectin-[D], collagen IV-[E], or gelatin-coated [F] plates displayed a more differentiated morphology within the first passage. Red arrows in [D-F] indicate that the differentiated colony consisted of dense centers containing cyst-like structures and necrotic cells. Laminin, therefore, appeared to be the minimal sufficient matrix protein.

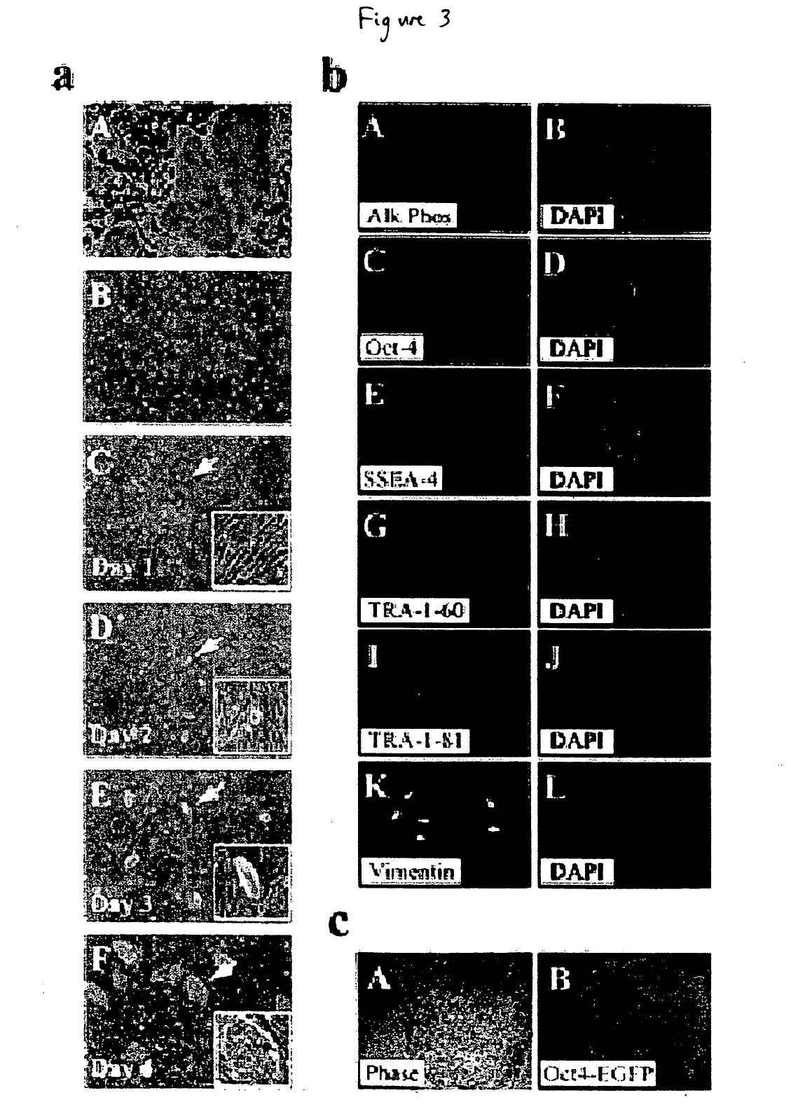

FIG. 3: Undifferentiated hESCs cultured under defined biologics-free conditions remain self-renewing following trypsin dissociation while creating a “self-contained”“self-supporting” system.

(a) Expanding hESCs clonally with trypsin treatment. hESCs maintained on laminin or laminin/collagen-coated plates in the hESC defined media were treated with trypsin [A] and dissociated into a single cell suspension [B]. These single cells were then allowed to seed on laminin or laminin/collagen-coated plates and cultivated in the defined hESC media containing 20 ng/ml bFGF [C-F]. Undifferentiated mature-sized hESC colonies were appeared after 4-7 days of culturing in the defined biologics-free conditions. Expansion into full Oct-4 positive colonies of individual cells (arrows) supports a conclusion of clonal self-renewal (see b). White arrows point to a single hESC [C, day 1 after seeding] and a single-cell-derived growing hESC colony [D-F, day 2-4 after seeding] that are shown at higher magnification in the inserts.

(b) Characterization hESCs passaged following trypsin-mediated dissociation. Immunofluorescence analysis indicates that hESCs inside the colonies that were maintained under the defined biologics-free culture condition and passaged by trypsin treatment for a prolonged period express the undifferentiated hESC markers alkaline phosphatase [A] (red), Oct-4 [C] (red), SSEA-4 [E] (red), Tra-1-60 [G] (red), and Tra-1-81 [I] (red). Cell colonies cease expressing those markers [A-J]. The colonies of undifferentiated cells appeared to be associated with a monolayer of hESC -derived fibroblastic cells that express vimentin [K] (red). White arrows in [K] delineate the edges of hESC colonies. Note that, in [K], the immunoreactive cells (the vimentin positive cells) are outside the colonies, presenting an image opposite to that shown in [A-J] where the immunopositive cells are within the colony. These “extra-colonial” differentiated cells may spontaneously act as “auto feeder layers” for the very same undifferentiated hESC colonies from which they were derived, preventing the latter from differentiating. The system now allowed these hESCs to produce their own support (“feeder”) cells. All cells in [A], [C], [E], [G], [I], and [K] are revealed by DAPI staining of their nuclei (blue) in [B], [D], [F], [H], [J], and [L], respectively.

(c) Undifferentiated hESCs carrying an Oct-4-driven reporter gene are capable of self-renewal under defined biologics-free conditions. Via a lentiviral vector, undifferentiated hESCs were transduced with a single copy of a reporter gene (enhanced green fluorescence protein [EGFP]) that is under the control of the Oct-4 promoter and cultivated under the feeder-free condition in the defined media containing 20 ng/ml bFGF for a prolonged period. A green [B] (Oct-4 expressing) undifferentiated hESC colony [A] subcloned from the infected cells is shown. [A] and [B] are images in the same field.

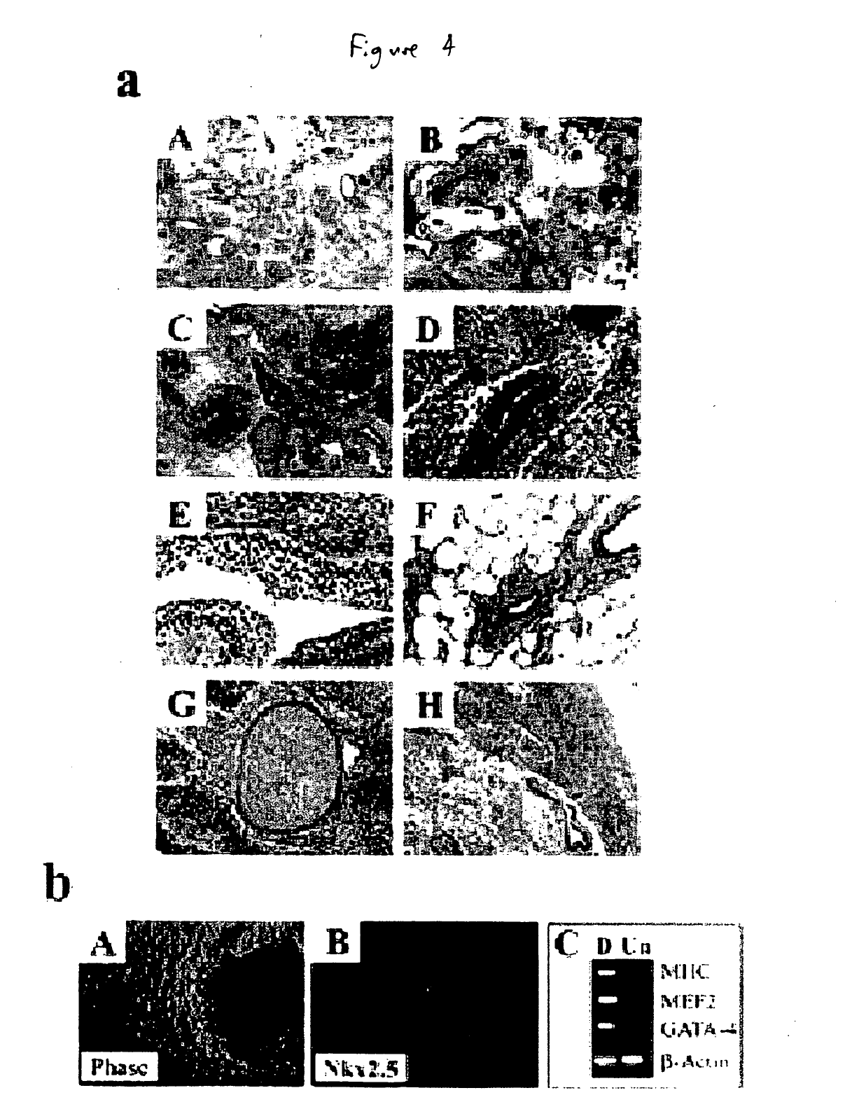

FIG. 4: Pluripotency of undifferentiated hESCs is sustained under the defined biologics-free conditions.

(a) Teratomas formed by hESCs cultured under defined biologics-free conditions. To assess their pluripotency, undifferentiated hESCs after prolonged propagation under the defined biologics-free conditions were injected into SCID mice. Histology analysis of teratomas generated in SCID mice revealed the presence of tissues of all three embryonic germ layers [A, 4X; B, 4X; and C, 10X], including pigmented neural tissue [D, 20X] (ectoderm); gut epithelium [E, 20X] (endoderm); adipose cells and blood vessels [F, 20X], cartilage [G, 20X], smooth muscle and connective tissue [H, 20X] (mesoderm).

(b) Cardiac differentiation of undifferentiated hESCs cultured under defined biologics-free conditions in vitro. Undifferentiated hESCs were detached and allowed to form EBs in a suspension culture. After permitting the EBs to attach to a gelatin-coated plate, “beating” cells [A] were observed in about one week. These beating cells expressed markers characteristic of cardiomyocytes, such as cardiac transcription factors Nkx2.5 [B] (by immunocytochemical analysis; the immunopositive cells in this panel are the same contracting cells in [A]), MEF-2, and GATA-4, as well as cardiac myosin heavy chain (MHC) (detected here by RT-PCR in the differentiated cells [“D”], but not in undifferentiated cells [“Un”]) [C]. These hESC -derived cardiomyocytes retained their contractility for >2 months.

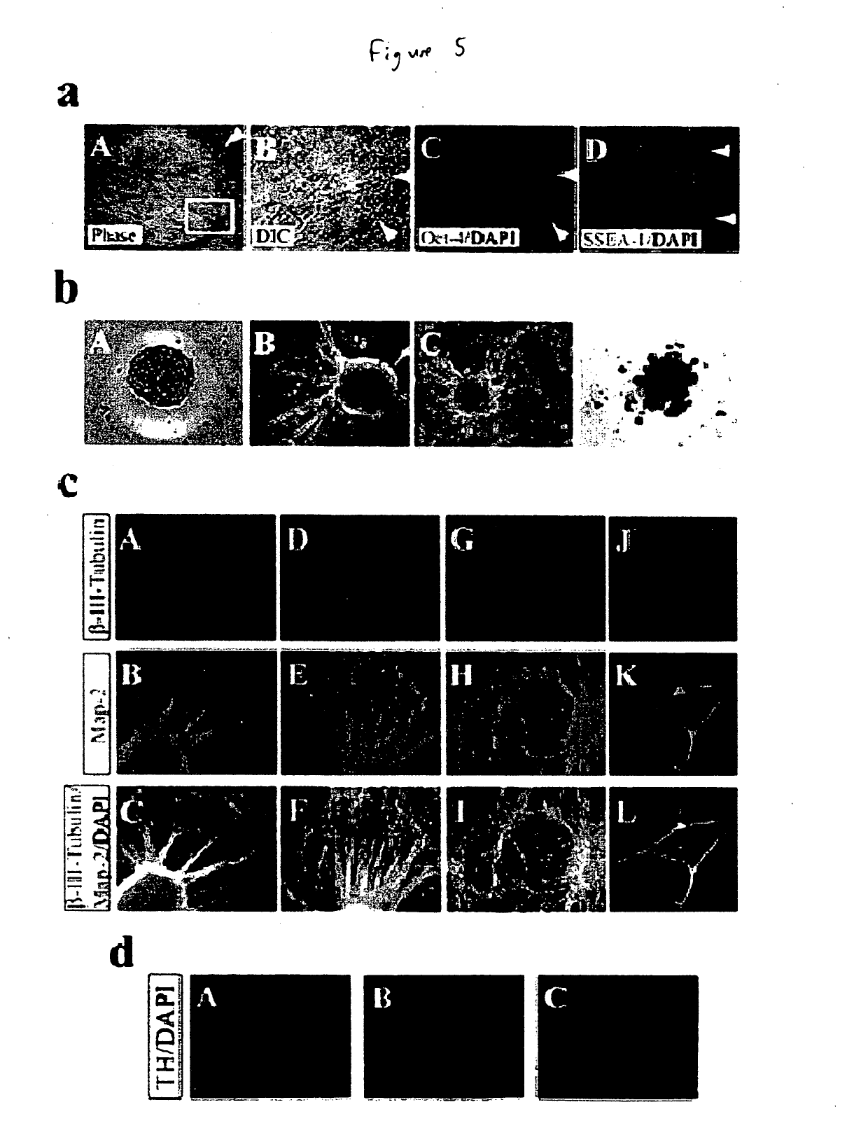

FIG. 5: Efficiently directing pluripotent hESCs cultured under “biologics”-free conditions towards a neuronal lineage.

(a) Retinoic acid was sufficient to induce differentiation of pluripotent hESCs maintained under serum-free, feeder-free, and conditioned-medium-free conditions. The hESC colonies cultured under the defined conditions described here began a differentiation “cascade” when treated with retinoic acid (10 μM) at the pluripotent undifferentiated stage, as indicated first by the emergence of a differentiated morphology (e.g., large cells) [A, B] and the expression of SSEA-1 [D] (red). These large differentiated cells inside the colonies ceased expressing Oct-4 [C] (red). However, these large cells continued to multiply and the colonies increased in size. The area delineated by the white square in [A] indicates the approximate area that is visualized at higher magnification in [B-D]. All cells in [C, D] are indicated by DAPI staining of their nuclei (blue).

(b) Generation of pigmented cells and process-bearing cells from differentiated hESCs in the biologics-free medium following induction by retinoic acid and the formation of cytospheres. The differentiated hESCs formed floating clusters of cells (cytospheres) when transferred to a suspension culture in serum-free media [A]. After permitting the clusters to attach to the surface of a tissue culture substrate—as occurs when bFGF is eliminated—there began to appear, after a week in culture, pigmented cells (with an appearance most consistent with those in the central nervous system) [B] (red arrow) as well as cells with extensive processes (resembling neurites) [B,C]. Isolated pigmented cells are shown at a higher magnification in [D]. (Note that the appearance of these monolayered, genuinely pigmented cells at high power is very different from the images created by extensively layered cells at low power as in FIG. 2d, A-D.)

(c) The process-bearing cells appear to be pursuing a neuronal phenotype. That the hESC -derived cells bearing the extensive network of processes were differentiating towards a neuronal lineage was suggested by their immunopositivity for the neuronal markers β-III-Tubulin (red) and MAP-2 (green) [A-I]. Single isolated hESC -derived neuronal cells expressing β-III-Tubulin and MAP-2 are shown in [J-L]. [C], [F], [I], and [L] are the merged images of [A] and [B], [D] and [E], [H] and [I], and [J] and [K], respectively. All cells in [C, F, I, L] are indicated by DAPI staining of their nuclei (blue).

(d) Tyrosine hydroxylase expression by some hESC -derived neuronal cells. A large subpopulation of these hESC -derived neuronal cells progressed to displaying expression of tyrosine hydroxylase (TH) [A-C] (red), suggesting the early stages of pursuing either a catecholaminergic or dopaminergic neurotransmitter phenotype. All cells are indicated by DAPI staining of their nuclei (blue). Importantly, note the co-presence of TH-negative cells in [B, C].

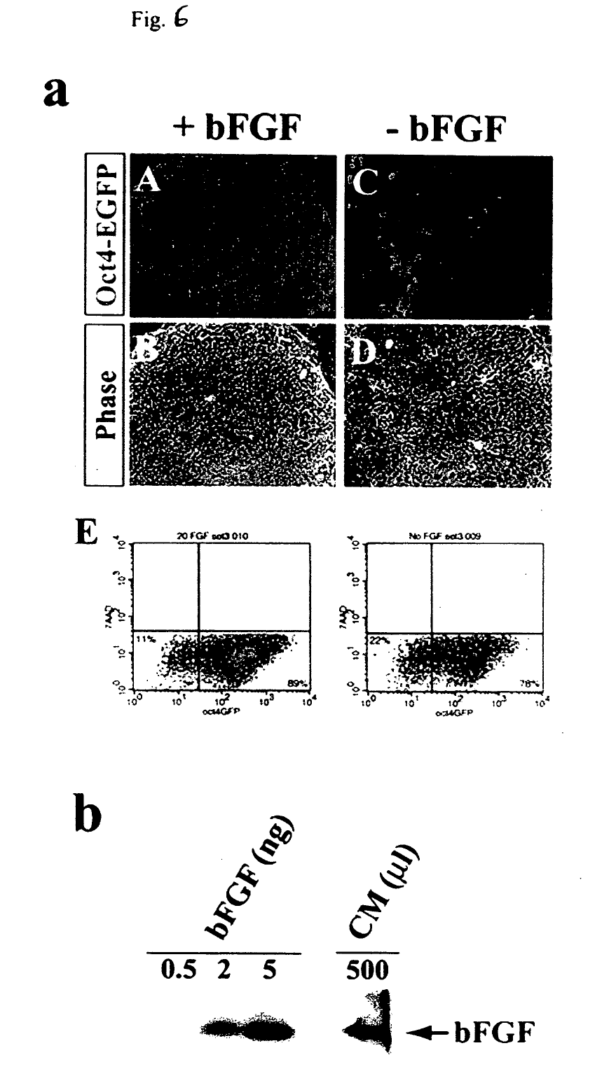

FIG. 6: (a) Growth of hESCs carrying an Oct-4-driven reporter gene in the presence or absence of bFGF. hESCs carrying a reporter gene (enhanced green fluorescence protein [EGFP]) that is under control of the Oct-4 promoter was generated via lentiviral-mediated transduction. Transfected hESCs were cultivated under the feeder-free conditions in the defined media with bFGF (20 ng/ml) or without bFGF for 4 days. In the presence of bFGF, hESCs displayed an undifferentiated morphology and a strong green fluorescence (Oct-4 expression) [A, B], while large, flattened, differentiated cells began to appear after 4 days of bFGF withdrawal with rapidly diminishing Oct-4 expression [C, D]. The cells were further dissociated and analyzed on a BD FacsSort instrument after staining with 7-aminoactinomycin D (7-AAD) to eliminate the dead cells [E]. With FACS-sorting, significant increases (200%) of Oct4-EGFP negative cells were observed after withdrawal bFGF for 4 days. (Note the actual percentage of Oct4-EGFP negative cells in the absence of bFGF should be even higher, because it takes around 2 days for the green fluorescence to be completely quenched after Oct-4 expression has been turned off.) (b) bFGF concentration in MEF-CM. Growth-arrested MEFs were obtained from Specialty Media (Phillipsburg, N.J.). MEFs were seeded at 56,000 cells/cm² in a gelatin-coated plate in 10% FBS/DMEM media for 24 hr., and then switched to hESC media for 24 hr. Conditioned medium was collected and concentrated with Ultrafree-15 centrifugal filter 5KNMWL (Millipore) for 10 folds. 50 μl of concentrates was loaded onto a SDS/PAGE gel and analyzed by Western blot. By using purified bFGF as standards, around 8-10 ng/ml endogenous bFGF was present in MEF-CM.

As those in the art will appreciate, the data and information represented in the attached figures is representative only and do not depict the full scope of the invention.

### DETAILED DESCRIPTION

1. Introduction

Before the present invention is described in detail, it is understood that the invention is not limited to the particular media compositions, culture systems, and methods described, as these may be readily adapted based on the descriptions provided herein. It is also to be understood that the terminology used herein is for the purpose of describing particular embodiments only, and is not intended to limit the scope of the invention defined by the appended claims.

2. Culturing Primate Stem Cells in a Substantially Undifferentiated State

This invention is based on the discovery of defined, isotonic cell culture media that can be used to culture stem cells, including primate primordial stem cells, particularly human embryonic stem cells, in a substantially undifferentiated state. The media is essentially serum-free, and does not require the use of a feeder cell layer or conditioned medium from separate cultures of feeder cells, although in some embodiments it is preferred to initially culture the stem cells in a growth environment that includes allogeneic feeder cells (or conditioned medium from such cells) prior to transferring the cells to fresh, feeder-free cultures for serial passaging (e.g., 1-50 or more passages). Given its defined nature, the media can be used to investigate the developmental effects of known growth factors and other compounds added exogenously to cultures of stem cells such as substantially undifferentiated primate primordial stem cells, including stem cells that have been genetically modified. It can also be used for many other applications, including (i) to screen for compounds that can direct the developmental fate of stem cells, for example, to further promote maintenance in culture of primate primordial stem cells in a substantially undifferentiated state or to induce differentiation toward a desired cell or tissue type, or to promote de-differentiation of a primate multipotent stem cell to a pluripotent stem cell, and (ii) to culture substantially undifferentiated human primordial stem cells for use in various cell therapy applications. A more thorough description of the invention and its applications appears below.

3. Culture Media

One aspect of the present invention provides a defined cell culture media for growing and maintaining stem cells, including primate-derived stem cells, particularly primate primordial stem cells, in a substantially undifferentiated state. In solution, the media are isotonic. In some embodiments, a medium has low osmotic pressure. The cell culture media of the invention includes a basal medium that is effective to support the growth of, for example, primate-derived primordial stem cells, and an amount of each of bFGF, insulin, and ascorbic acid necessary to support substantially undifferentiated growth of the stem cells. Preferably, the bFGF and insulin used are produced by recombinant methods, although they may be isolated from natural sources. Also, preferably the protein used is from the same primate species as the stem cells to be cultured. With regard to the bFGF and insulin proteins, the invention also contemplates the use of homologs, or proteins having sequence identity of at least about 70% and the receptor activating activity of the respective naturally occurring protein (i.e., bFGF or insulin, as the case may be), artificial analogs, polypeptide fragments that activate the respective bFGF or insulin receptor and/or downstream signaling, and other molecules that activate the bFGF or insulin receptors and/or their downstream signaling. Thus, for purposes of the invention, a molecule that activates the bFGF receptor and/or its downstream signaling in an analogous fashion to bFGF (even with greater or reduced effectiveness, for example having at least 25%, at least 50%, at least 75%, at least 100%, at least 150%, at least 300%, at lest 500%, or at least 5000% of activation activity per molecule as compared to the naturally occurring bFGF protein) shall be considered “bFGF ”, provided that it can be used in lieu of the bFGF protein in a defined cell culture media for growing and maintaining primate primordial stem cells in a substantially undifferentiated state. Similarly, a molecule that activates the insulin receptor and/or its downstream signaling in an analogous fashion to insulin (even with greater or reduced effectiveness) shall be considered “insulin,” provided that it can be used in lieu of the insulin protein in a defined cell culture media for growing and maintaining primate primordial stem cells in a substantially undifferentiated state, for example having at least 25%, at least 50%, at least 75%, at least 100%, at least 150%, at least 300%, at lest 500%, or at least 5000% of activation activity per molecule as compared to the naturally occurring insulin protein. With regard to ascorbic acid, the invention envisions the use of any other molecule, including any derivative or analogue of ascorbic acid, which exhibits activity analogous to that observed for ascorbic acid when used in the defined media of the invention. Here, “analogous” does not require an equivalent level of activity per molecule of bFGF, insulin, or ascorbic acid and another molecular species having the particular activity in the defined media of the invention. Thus, different amounts of the molecular species substituted for bFGF, insulin, and/or ascorbic acid may be required to obtain the same biological effect as achieved using bFGF, insulin, and/or ascorbic acid, as the case may be. As will be appreciated, molecules that can be substituted for bFGF, insulin, or ascorbic acid, as the case may be, are “functional equivalents” of the molecules for which they are substituted, even if different amounts of the functionally equivalent molecules are required to achieve the same results as can be obtained using a naturally occurring form of bFGF, insulin, or ascorbic acid.

A medium according to the invention may also include, without limitation, non-essential amino acids, an anti-oxidant, a reducing agent, growth factors, and a pyruvate salt. The base media may, for example be Dulbecco's Modified Eagle Medium (DMEM), DMEM/F-12, or KO-DMEM, each supplemented with L-glutamine or GlutaMAX™-I (provided as the dipeptide L-alanyl-L-glutamine (Invitrogen) at a final concentration of 2 mM), non-essential amino acids (1%), and 100 μM β-mercaptoethanol. A medium is preferably sterilized (e.g., by filtration) prior to addition to a cell culture.

Table 1 below sets forth a representative example of a basal medium based on DMEM that can be used in practicing the invention. Other basal media useful in mammalian cell culture include, without limitation, Basal Media Eagle (BME), Glasgow Minimum Essential Media, Iscove's Modified Dulbecco's Media, Minimum Essential Media (MEM), Modified Eagle Medium (MEM), Opti-MEM I Reduced Serum Media, RPMI Media 1640, Waymouth's MB 752/1 Media, Williams Media E, Medium NCTC-109, neuroplasma medium, BGJb Medium, Brinster's BMOC-3 Medium, CMRL Medium, CO2-Independent Medium, Leibovitz's L-15 Media, McCoy's 5A Media (modified), and MCDB 131 Medium.

TABLE 1
Representative Base Medium (based on
Dulbecco's Modified Eagle's Medium)

| Description | mg/L | |
|---|---|---|
| CaCl₂ (anhydrous) | 200.0 | Inorganic salts |
| Fe(NO₃) · 9H₂O | 0.1 | |
| KCl | 400.0 | |
| MgSO₄ (anhydrous) | 97.7 | |
| NaCl | 6400.0 | |
| NaH₂PO₄ · H₂O | 125.0 | |
| L-Arginine HCl | 84.0 | Amino Acids |
| L-Cystine 2HCl | 62.6 | |
| L-Glutamine | 584.0 | |
| Glycine | 30.0 | |
| L-Histidine HCl · H₂O | 42.0 | |
| L-Isoleucine | 104.8 | |
| L-Leucine | 104.8 | |
| L-Lysine HCl | 146.2 | |
| L-Methionine | 30.0 | |
| L-Phenylalanine | 66.0 | |
| L-Serine | 42.0 | |
| L-Threonine | 95.2 | |
| L-Tryptophan | 16.0 | |
| L-Tryosine 2Na · 2H₂O | 103.8 | |
| L-Valine | 93.6 | |
| D-Ca Pantothenic Acid | 4.0 | Vitamins |
| Choline Chloride | 4.0 | |
| Folic Acid | 4.0 | |
| Myo-Inositol | 7.0 | |
| Niacinamide | 4.0 | |
| Pyridoxal HCl | 4.0 | |
| Pyridoxine HCl | 4.0 | |
| Riboflavin | 0.4 | |
| Thiamine HCl | 4.0 | |
| D-Glucose | 4500.0 | Other |
| Phenol Red (Sodium) | 15.9 | |
| Sodium Pyruvate | 110.0 | |
| Add NaHCO₃ | 1500-3700 | |

Exogenous growth factors may also be added to a medium according to the invention to assist in the maintenance of cultures of stem cells (e.g., primate primordial stem cells) in a substantially undifferentiated state. Such factors and their effective concentrations can be identified as described elsewhere herein or using techniques known to those of skill in the art of culturing cells. Representative examples of growth factors useful in this regard include bFGF, insulin, acidic FGF (aFGF), epidermal growth factor (EGF), insulin-like growth factor I (IGF-I), IGF-II, platelet-derived growth factor (PDGF), and vascular endothelial growth factor (VEGF), activin-A, bone morphogenic proteins (BMPs), forskolin, glucocorticords (e.g., dexamethasone), transferrins, and albumins.

Useful reducing agents include P-mercaptoethanol. In a preferred embodiment, the β-mercaptoethanol is present in a concentration of about 0.1 mM. Other reducing agents such as monothioglycerol or dithiothreitol (DTT), alone or in combination, can be used to similar effect. Still other equivalent substances will be familiar to those of skill in the cell culturing arts.

Pyruvate salts may also be included in a medium according to the invention. Pyruvate salts include sodium pyruvate or another pyruvate salt effective maintaining and/or enhancing primate primordial stem cell growth in a substantially undifferentiated state such as, for example, potassium pyruvate. In preferred embodiments, the pyruvate salt is added to a concentration of about 0.1 mM.

Other compounds suitable for supplementing a culture medium of the invention include nucleosides (e.g., adenosine, cytidine, guanosine, uridine, and thymidine) and nucleotides. Nucleosides and/or nucleotides can be included in a variety of concentrations, preferably ranging from about 0.1 μM (micromolar) to about 50 μM.

In preferred embodiments, a medium's endotoxicity, as measured in endotoxin units per milliliter (“eu/ml”), will be less than about 0.1 eu, and, in more preferred embodiments, will be less than about 0.05 eu/ml. In particularly preferred embodiments, the endotoxicity of the base medium will be less than about 0.03 eu/ml. Methods for measuring endotoxicity are known in the art. For example, a preferred method is described in the “Guideline on Validation of the Limulus Amebocyte Lysate Test as an End-product Endotoxin Test for Human and Animal Parental Drugs, Biological Products and Medical Devices,” published by the U.S. Department of Health and Human Services, FDA, December 1987.

As will be appreciated, it is desirable to replace spent culture medium with fresh culture medium either continually, or at periodic intervals, preferably every 1 to 3 days. One advantage of using fresh medium is the ability to adjust conditions so that the cells expand more uniformly and rapidly than they do when cultured on feeder cells according to conventional techniques, or in conditioned medium.

Populations of stem cells (such as primate primordial stem cells) can be obtained that are 4-, 10-, 20-, 50-, 100-, 1000-, or more fold expanded when compared to the previous starting cell population. Under suitable conditions, cells in the expanded population will be 50%, 70%, or more in the undifferentiated state, as compared to the stem cells used to initiate the culture. The degree of expansion per passage can be calculated by dividing the approximate number of cells harvested at the end of the culture by the approximate number of cells originally seeded into the culture. Where geometry of the growth environment is limiting or for other reasons, the cells may optionally be passaged into a similar growth environment for further expansion. The total expansion is the product of all the expansions in each of the passages. Of course, it is not necessary to retain all the expanded cells on each passage. For example, if the cells expand two-fold in each culture, but only about 50% of the cells are retained on each passage, then approximately the same number of cells will be carried forward. But after four cultures, the cells are said to have undergone an expansion of 16-fold. Cells that are not passaged forward may be retained, if desired, in which event they may be frozen and stored, preferably in liquid nitrogen or at −140° C.

Of course, culture conditions inappropriate for stem cells such as primate primordial stem cells will cause the cells to differentiate promptly, although it will be appreciated that marginally beneficial conditions may allow the stem cells to go through a few passages while still retaining a proportion of undifferentiated cells. In order to test whether conditions are adequate for indefinite culture of stem cells (e.g., primate primordial stem cells) in a substantially undifferentiated state, it is recommended that the cells be expanded in a preferable range of about 4- to about 10-fold every passage. A higher degree of expansion and/or a higher number of passages (e.g., at least 11 passages) provides a more rigorous test. An effective test for whether a cell population is substantially undifferentiated is the demonstration that the cells express cell surface markers indicative of an undifferentiated state.

4. Primate-Derived Primordial Stem Cells

Stem cells, including primate primordial stem cells, cultured in accordance with the invention can be obtained from any suitable source using any appropriate technique. For example, procedures for isolating and growing human primordial stem cells are described in U.S. Pat. No. 6,090,622. Procedures for obtaining Rhesus monkey and other non-human primate primordial stem cells are described in U.S. Pat. No. 5,843,78 and international patent publication WO 96/22362. In addition, methods for isolating Rhesus monkey primordial stem cells are described by Thomson, et al. ((1995), Proc. Natl. Acad. Sci. USA, vol. 92:7844-7848).

Human embryonic stem cells (hESCs) can be isolated, for example, from human blastocysts obtained from human in vivo preimplantation embryos, in vitro fertilized embryos, or one-cell human embryos expanded to the blastocyst stage (Bongso, et al. (1989), Hum. Reprod., vol. 4: 706). Human embryos can be cultured to the blastocyst stage in G1.2 and G2.2 medium (Gardner, et al. (1998), Fertil. Steril., vol. 69:84). The zona pellucida is removed from blastocysts by brief exposure to pronase (Sigma). The inner cell masses can be isolated by immunosurgery or by mechanical separation, and are plated on mouse embryonic feeder layers, or in the defined culture system as described herein. After nine to fifteen days, inner cell mass-derived outgrowths are dissociated into clumps either by exposure to calcium and magnesium-free phosphate-buffered saline (PBS) with 1 mM EDTA, by exposure to dispase, collagenase, or trypsin, or by mechanical dissociation with a micropipette. The dissociated cells are then replated as before in fresh medium and observed for colony formation. Colonies demonstrating undifferentiated morphology are individually selected by micropipette, mechanically dissociated into clumps, and replated. Embryonic stem cell-like morphology is characterized as compact colonies with apparently high nucleus to cytoplasm ratio and prominent nucleoli. Resulting embryonic stem cells are then routinely split every 1-2 weeks by brief trypsinization, exposure to Dulbecco's PBS (without calcium or magnesium and with 2 mM EDTA), exposure to type IV collagenase (about 200 U/mL), or by selection of individual colonies by mechanical dissociation, for example, using a micropipette.

Once isolated, the stem cells, e.g., primate stem cells, can be cultured in a culture medium according to the invention that supports the substantially undifferentiated growth of primate primordial stem cells using any suitable cell culturing technique. For example, a matrix laid down prior to lysis of primate feeder cells (preferably allogeneic feeder cells) or a synthetic or purified matrix can be prepared using standard methods. The primate primordial stem cells to be cultured are then added atop the matrix along with the culture medium. In other embodiments, once isolated, undifferentiated human embryonic stem cells can be directly added to an extracellular matrix that contains laminin or a growth-arrested human feeder cell layer (e.g., a human foreskin fibroblast cell layer) and maintained in a serum-free growth environment according to the culture methods of invention. Unlike existing human embryonic stem cell lines cultured using conventional techniques, human embryonic stem cells and their derivatives prepared and cultured in accordance with the instant methods can be used therapeutically since they will not have been exposed to animal feeder cells, feeder-cell conditioned media, or serum at some point of their life time, thereby avoiding the risks of contaminating human cells with non-human animal cells, transmitting pathogens from non-human animal cells to human cells, forming heterogeneous fusion cells, and exposing human cells to toxic xenogeneic factors.

Alternatively, the stem cells, e.g., primate primordial stem cells, can be grown on living feeder cells (preferably allogeneic feeder cells) using methods known in the cell culture arts. The growth of the stem cells is then monitored to determine the degree to which they have become differentiated, for example, using a marker for alkaline phosphatase or telomerase or detecting the expression of the transcription factor Oct-4, or by detecting a cell surface marker indicative of an undifferentiated state (e.g., in the context of human embryonic stem cells, a labeled antibody for any one or more of SSEA-4, Tra-1-60, and Tra-1-81). When the culture has grown to confluence, at least a portion of the undifferentiated cells is passaged to another culture vessel. The determination to passage the cells and the techniques for accomplishing such passaging can be performed in accordance with the culture methods of invention (e.g., through morphology assessment and dissection procedures).

In certain preferred embodiments, the cells are cultured in a culture vessel that contains a substrate selected from the group consisting of feeder cells, preferably allogeneic feeder cells, an extracellular matrix, a suitable surface and a mixture of factors that adequately activate the signal transduction pathways required for undifferentiated growth, and a solution-borne matrix sufficient to support growth of the stem cells in solution. Thus, in addition to the components of the solution phase of culture media of the invention, the growth environment includes a substrate selected from the group consisting of primate feeder cells, preferably allogeneic feeder cells, and an extracellular matrix, particularly laminin. Preferred feeder cells for primate primordial stem cells include primate fibroblasts and stromal cells. In preferred embodiments, the feeder cells and stem cells are allogeneic. In the context of human embryonic stem cells, particularly preferred feeder cells include human fibroblasts, human stromal cells, and fibroblast-like cells derived from human embryonic stem cells. If living feeder cells are used, as opposed to a synthetic or purified extracellular matrix or a matrix prepared from lysed cells, the cells can be mitotically inactivated (e.g., by irradiation or chemically) to prevent their further growth during the culturing of primate primordial stem cells. Inactivation is preferably performed before seeding the cells into the culture vessel to be used. The primate primordial stem cells can then be grown on the plate in addition to the feeder cells. Alternatively, the feeder cells can be first grown to confluence and then inactivated to prevent their further growth. If desired, the feeder cells may be stored frozen in liquid nitrogen or at −140° C. prior to use. As mentioned, if desired such a feeder cell layer can be lysed using any suitable technique prior to the addition of the stem cells (e.g., primate stem cells) so as to leave only an extracellular matrix.

Not wishing to be bound to any theory, it is believed that the use of such feeder cells, or an extracellular matrix derived from feeder cells, provides one or more substances necessary to promote the growth of stem cells (e.g., primate primordial stem cells) and/or prevent or decrease the rate of differentiation of such cells. Such substances are believed to include membrane-bound and/or soluble cell products that are secreted into the surrounding medium by the feeder cells. Thus, those skilled in the art will recognize that additional cell lines can be used with the cell culture media of the present invention to equivalent effect, and that such additional cell lines can be identified using standard methods and materials, for example, by culturing over time (e.g., several passages) substantially undifferentiated primate primordial stem cells on such feeder cells in a culture medium according to the invention and determining whether the stem cells remain substantially undifferentiated over the course of the analysis. Also, because of the defined nature of the culture media provided herein, it is now possible to assay various compounds found in the extracellular matrix or secreted by feeder cells to determine their respective roles in the growth, maintenance, and differentiation of stem calls such as primate primordial stem cells.

When purified components from extracellular matrices are used in lieu of feeder cells, such components will include those provided by the extracellular matrix of a suitable feeder cell layer. Components of extracellular matrices that can be used include laminin, or products that contain laminin, such as MATRIGEL®, or other molecules that activate the laminin receptor and/or its downstream signaling pathway. Thus, for purposes of the invention, a molecule that activates the laminin receptor and/or its downstream signaling pathway in an analogous fashion to laminin (even with greater or reduced effectiveness, for example, having at least 25%, at least 50%, at least 75%, at least 100%, at least 150%, at least 300%, at lest 500%, or at least 5000% of activation activity per molecule as compared to a naturally occurring or recombinant form of laminin) shall be considered “laminin”provided that it can be used in lieu of the laminin in a defined cell culture media for growing and maintaining primate primordial stem cells in a substantially undifferentiated state. MATRIGEL® is a soluble preparation from Engelbreth-Holm-Swarm tumor cells that gels at room temperature to form a reconstituted basement membrane. Other extracellular matrix components include fibronectin, collagen, and gelatin. In addition, one or more substances produced by the feeder cells, or contained in an extracellular matrix produced by a primate feeder cell line, can be identified and used to make a substrate that obviates the need for feeder cells. Alternatively, these components can be prepared in soluble form so as to allow the growth and maintenance of undifferentiated of stem cells in suspension culture. Thus, this invention contemplates adding extracellular matrix to the fluid phase of a culture at the time of passaging the cells or as part of a regular feeding, as well as preparing the substrate prior to addition of the fluid components of the culture.

Any suitable culture vessel can be adapted to culture stem cells (e.g., primate primordial stem cells) in accordance with the invention. For example, vessels having a substrate suitable for matrix attachment include tissue culture plates (including multi-well plates), pre-coated (e.g., gelatin—pre-coated) plates, T-flasks, roller bottles, gas permeable containers, and bioreactors. To increase efficiency and cell density, vessels (e.g., stirred tanks) that employ suspended particles (e.g., plastic beads or other microcarriers) that can serve as a substrate for attachment of feeder cells or an extracellular matrix can be employed. In other embodiments, undifferentiated stem cells can be cultured in suspension by providing the matrix components in soluble form. As will be appreciated, fresh medium can be introduced into any of these vessels by batch exchange (replacement of spent medium with fresh medium), fed-batch processes (i.e., fresh medium is added without removal of spent medium), or ongoing exchange in which a proportion of the medium is replaced with fresh medium on a continuous or periodic basis.

5. Applications

The defined cell culture media and methods for growing stem cells, particularly primate primordial stem cells, in a substantially undifferentiated state in accordance with the present invention will be seen to be applicable to all technologies for which stem cell lines are useful. Of particular importance is the use of the instant cell culture media and methods of culturing, for example, primate primordial stem cells in screening to identify growth factors useful in culturing primate stem cells in an undifferentiated state, as well as compounds that induce such cells to differentiate toward a particular cell or tissue lineage. The instant invention also allows genetically modified stem cells to be developed, as well as the creation of new stem cell lines, especially new primate primordial stem cell lines. The establishment of new cell lines according to the invention includes normal stem cell lines, as well as abnormal stem cell lines, for example, stem cell lines that carry genetic mutations or diseases (e.g., stem cells infected with a pathogen such as a virus, for example, HIV). Cells produced using the media and methods of the invention can also be mounted on surfaces to form biosensors for drug screening. The invention also provides for the capacity to produce, for example, commercial grade undifferentiated primate primordial stem cells (e.g., human ESCs) on a commercial scale. As a result, stem cells such as primate primordial stem cells produced in accordance with the present invention will have numerous therapeutic and diagnostic applications. In other applications, substantially undifferentiated hESCs can be used. Several representative examples of such applications are provided below.

A. Screens for Growth Factors

An aspect of the present invention involves screens for identifying growth factors that promote or inhibit the differentiation, growth, or survival of stem cells such as primate primordial stem cells in serum-free, feeder-free culture, as well as factors that promote the differentiation of such cells. Such systems have the advantage of not being complicated by secondary effects caused by perturbation of the feeder cells by the test compounds. In some embodiments, primate primordial stem cells are used as a primary screen to identify substances that promote the growth of primate primordial stem cells in a substantially undifferentiated state. Such screens are performed by contacting the stem cells in culture with one test compound species (or, alternatively, pools of different test compounds). The effect of exposing the cells to the test compound can then be assessed using any suitable assay, including enzyme activity-based assays and reporter/antibody-based screens, e.g., to detect the presence of a marker correlated with an undifferentiated state. Such assays can be either qualitative or quantitative in terms of their read out. Suitable enzyme activity assays are known in the art (e.g., assays based on alkaline phosphatase or telomerase activity), as are antibody-based assays, any of which may readily be adapted for such applications. Of course, any other suitable assay may also be employed, depending on the result being sought.

With regard to antibody-based assays, polyclonal or monoclonal antibodies may be obtained that are specifically reactive with a cell surface marker that is correlated with totipotency or pluripotency. Such antibodies can be labeled. Alternatively, their presence may be detected by a labeled secondary antibody (e.g., a fluorescently labeled, rabbit-derived anti-mouse antibody that reacts with mouse-derived antibodies), as in a standard ELISA (Enzyme-Linked ImmunoSorbent Assay). If desired, labeled stem cells can also be sorted and counted using standard methods, e.g., fluorescence-activated cell sorting (“FACS”).

In one embodiment of such a primary screen, the presence of increased alkaline phosphatase activity (indicative of an undifferentiated state) indicates that the test compound is a growth factor. In other embodiments, increased percentages of cells with continued expression of one or more markers indicative of an undifferentiated state (e.g., Oct-4, SSEA-4, Tra-1-60, and Tra-1-81) following exposure to a test compound indicates that the test compound is a growth factor. Serial or parallel combinations of such screens (e.g., an alkaline phosphatase-based screen followed by, or alternatively coupled with, a screen based on expression of Oct-4, SSEA-4, Tra-1-60, and Tra-1-81) may also be employed. Substances that are found to produce statistically significant promotion of growth of the stem cells in an undifferentiated state can then be re-tested, if desired. They can also be tested, for example, against primordial stem cells from other primate species to determine if the growth factor exerts only species-specific effects. Substances found to be effective growth factors for primate stem cells can also be tested in combinations to determine the presence of any synergistic effects.

Such assays can also be used to optimize the culture conditions for a particular type of stem cell, such as primate primordial stem cells (e.g., human ESCs).

In addition to screening for growth factors, stem cells cultured in accordance with the invention can also be used to identify other molecules useful in the continued culture of the cells in a substantially undifferentiated state, or alternatively, which stimulate a change in the developmental fate of a cell. Such changes in developmental fate include inducing differentiation of the stem cell toward a desired cell lineage. In other embodiments, the developmental change stimulated by the molecule may be de-differentiation, such that following exposure to the test compound, the cells become more primitive, in that subsequent to exposure, they have the capacity to differentiate into more cell types than was possible prior to exposure. As will be appreciated, such methods allow the evaluation of any compound for such an effect, including compounds already known to play important roles in biology, e.g., proteins, carbohydrates, lipids, and various other organic and inorganic molecules found in cells or which affect cells.

B. Drug Screens

Feeder-free, serum-free cultures of stem cells such as primate primordial stem cells can also be used in drug discovery processes, as well as for testing pharmaceutical compounds for potential unintended activities, as might cause adverse reactions if the compound was administered to a patient. Assessment of the activity of pharmaceutical test compounds generally involves combining the cells of the invention with the test compound, determining any resulting change, and then correlating the effect of the compound with the observed change. The screening may be done, for example, either because the compound is designed to have a pharmacological effect on certain cell types, or because a compound designed to have effects elsewhere may have unintended side effects. Two or more drugs (or other test compounds) can also be tested in combination (by combining with the cells either simultaneously or sequentially) to detect possible drug-drug interaction effects. In some applications, compounds are screened initially for potential toxicity. See generally “In vitro Methods in Pharmaceutical Research,” Academic Press, 1997. Cytotoxicity can be determined by the effect on cell viability, survival, morphology, on the expression or release of certain markers, receptors or enzymes, and/or on DNA synthesis or repair, measured by [³H]-thymidine or BrdU incorporation.

C. Differentiated Cells

Primate primordial stem cells (or other stem cells) cultured according to this invention can be used to prepare populations of differentiated cells of various commercially and therapeutically important tissue types. In general, this is accomplished by expanding the stem cells to the desired number. Thereafter, they are caused to differentiate according to any of a variety of differentiation strategies. For example, highly enriched populations of cells of the neural lineage can be generated by changing the cells to a culture medium containing one or more neurotrophins (such as neurotrophin 3 or brain-derived neurotrophic factor), one or more mitogens (such as epidermal growth factor, bFGF, PDGF, IGF 1, and erythropoietin), or one or more vitamins (such as retinoic acid, ascorbic acid). Alternatively, multipotent neural stem cells can be generated through the embryoid body stage and maintained in a chemically defined medium containing bFGF. Cultured cells are optionally separated based on whether they express a nerve precursor cell marker such as nestin, Musashi, vimentin, A2B5, nurrl, or NCAM. Using such methods, neural progenitor/stem cells can be obtained having the capacity to generate both neuronal cells (including mature neurons) and glial cells (including astrocytes and oligodendrocytes). Alternatively, replicative neuronal precursors can be obtained that have the capacity to form differentiated cell populations.

Cells highly enriched for markers of the hepatocyte lineage can be differentiated from primate primordial stem cells by culturing the stem cells in the presence of a histone deacetylase inhibitor such as n-butyrate. The cultured cells are optionally cultured simultaneously or sequentially with a hepatocyte maturation factor such as EGF, insulin, or FGF.

Primate primordial stem cells can also be used to generate cells that have characteristic markers of cardiomyocytes and spontaneous periodic contractile activity. Differentiation in this way is facilitated by nucleotide analogs that affect DNA methylation (such as 5-aza-deoxy-cytidine), growth factors, and bone morphogenic proteins. The cells can be further enriched by density-based cell separation, and maintained in media containing creatine, carnitine, and taurine.

Additionally, stem cells such as primate primordial stem cells can be directed to differentiate into mesenchymal cells in a medium containing a bone morphogenic protein (BMP), a ligand for the human TGF-β receptor, or a ligand for the human vitamin D receptor. The medium may further comprise dexamethasone, ascorbic acid-2-phosphate, and sources of calcium and phosphate. In preferred embodiments, derivative cells have phenotypic features of cells of the osteoblast lineage.

As will be appreciated, differentiated cells derived from stem cells such as primate primordial stem cells cultured in accordance with the methods of the invention can be also be used for tissue reconstitution or regeneration in a human patient in need thereof. The cells are administered in a manner that permits them to graft to the intended tissue site and reconstitute or regenerate the functionally deficient area. For instance, neural precursor cells can be transplanted directly into parenchymal or intrathecal sites of the central nervous system, according to the disease being treated. The efficacy of neural cell transplants can be assessed in a rat model for acutely injured spinal cord, as described by McDonald, et al. ((1999) Nat. Med., vol. 5:1410) and Kim, et al. ((2002) Nature, vol. 418:50). Successful transplants will show transplant-derived cells present in the lesion 2-5 weeks later, differentiated into astrocytes, oligodendrocytes, and/or neurons; and migrating along the spinal cord from the lesioned end, and an improvement in gait, coordination, and weight-bearing.

Similarly, the efficacy of cardiomyocytes can be assessed in a suitable animal model of cardiac injury or dysfunction, e.g., an animal model for cardiac cryoinjury where about 55% of the left ventricular wall tissue becomes scar tissue without treatment (Li, et al. (1996), Ann. Thorac. Surg., vol. 62:654; Sakai, et al. (1999), Ann. Thorac. Surg., vol. 8:2074; Sakai, et al. (1999), J. Thorac. Cardiovasc. Surg., vol. 118:715). Successful treatment will reduce the area of the scar, limit scar expansion, and improve heart function as determined by systolic, diastolic, and developed pressure (Kehat, et al. (2004)). Cardiac injury can also be modeled, for example, using an embolization coil in the distal portion of the left anterior descending artery (Watanabe, et al. (1998), Cell Transplant., vol. 7:239), or by ligation of the left anterior descending coronary artery (Min, et al. (2002), J. Appl. Physiol., vol. 92:288). Efficacy of treatment can be evaluated by histology and cardiac function. Cardiomyocyte preparations embodied in this invention can be used in therapy to regenerate cardiac muscle and treat insufficient cardiac function.

Liver function can also be restored by administering hepatocytes and hepatocyte precursors differentiated from, for example, primate pluripotent stem cells grown in accordance with this invention. These differentiated cells can be assessed in animal models for ability to repair liver damage. One such example is damage caused by intraperitoneal injection of D-galactosamine (Dabeva, et al. (1993), Am. J. Pathol., vol. 143:1606). Treatment efficacy can be determined by immunocytochemical staining for liver cell markers, microscopic determination of whether canalicular structures form in growing tissue, and the ability of the treatment to restore synthesis of liver-specific proteins. Liver cells can be used in therapy by direct administration, or as part of a bioassist device that provides temporary liver function while the subject's liver tissue regenerates itself, for example, following fulminant hepatic failure.

D. Genetically Modified Primate Stem Cells

The present invention also provides methods for producing, for example, primate stem cell lines having one or more genetic modifications. As is apparent to one of ordinary skill in the art, altered expression of gene products can be achieved by modifying the coding sequence of a gene product or by altering flanking regions of the coding sequence. Thus, as used herein, the terms “genetic modification” and the like include alterations to the sequence encoding a gene product, as well as alterations to flanking regions, in particular to the 5′ upstream region of the coding sequence (including the promoter). Similarly, the term “gene” encompasses all or part of the coding sequence and the regulatory sequences that may be present flanking the coding sequence, as well as other sequences flanking the coding sequence. Genetic modifications may be permanent or transient. Preferred permanent modifications are those that do not adversely affect chromosome stability or cell replication. Such modifications are preferably introduced by recombination or otherwise by insertion into a chromosome (as may be mediated, for example, by an engineered retroviral vector). Transient modifications are generally obtained by introducing an extrachromosomal genetic element into a cell by any suitable technique. Regardless of the permanence of a particular genetic modification, in embodiments wherein one or more genes are introduced, their expression may be inducible or constitutive. The design, content, stability, etc. of a particular genetic construct made for use in practicing the invention is left to the discretion of the artisan, as these will vary depending on the intended result.

After introducing a desired genetic modification, a particularly effective way of enriching genetically modified cells is positive selection using resistance to a drug such as neomycin. To accomplish this, the cells can be genetically altered by contacting them simultaneously with a vector system harboring the gene(s) of interest and a vector system that provides the drug resistance gene. Alternatively, the drug resistance gene can be built into the same vector as the gene(s) of interest. After transfection has taken place, the cultures are treated with the corresponding drug, and untransfected cells are eliminated.

According to this aspect, genetically modified stem cells such as primate primordial stem cells are grown using a cell culture medium of the invention. One or more genes or nucleic acid molecules are introduced into, or one or more genes are modified in, these cells to produce a clone population having the desired genetic modifications. Depending upon the genetic modification(s) made, the cells may continue to be propagated in a substantially undifferentiated state in accordance with the invention. Alternatively, they may be allowed (or induced) to differentiate. Primate-derived primordial stem cells having such genetic modifications have important applications, especially with respect to applications where euploid primate cells having genetic modifications are useful or required. Examples of such applications include, but are not limited to, the development of cell-based models for primate, especially human, diseases, as well as the development of specialized tissues for transplantation. Genetically modified stem cells cultured in accordance with the invention, including primate primordial stem cells, especially human embryonic stem cells, also have many other therapeutic applications, including in gene therapy (e.g., to compensate for a single gene defect), and as tissue for grafting or implantation, and to treat other diseases and disorders. Examples of diseases caused by single gene defects include myotonic dystrophy, cystic fibrosis, sickle cell anemia, Tay Sachs disease, and hemophilia.

For therapeutic application, cells prepared according to this invention (be they totipotent or pluripotent cells or differentiated cells derived therefrom) are typically supplied in the form of a pharmaceutical composition comprising an isotonic excipient, and are prepared under conditions that are sufficiently sterile for human administration. For general principles in medicinal formulation of cell compositions, see “Cell Therapy: Stem Cell Transplantation, Gene Therapy, and Cellular Immunotherapy,” by Morstyn & Sheridan eds, Cambridge University Press, 1996; and “Hematopoietic Stem Cell Therapy,” E. D. Ball, J. Lister & P. Law, Churchill Livingstone, 2000. The cells may be packaged in a device or container suitable for distribution or clinical use, optionally accompanied by information relating to use of the cells in tissue regeneration or for restoring a therapeutically important metabolic function.

### EXAMPLES

The following Examples are provided to illustrate certain aspects of the present invention and to aid those of skill in the art in practicing the invention. These Examples are in no way to be considered to limit the scope of the invention in any manner.

General methods in molecular genetics and genetic engineering are described in the current editions of “Molecular Cloning: A Laboratory Manual” (Sambrook, et al., Cold Spring Harbor); Gene Transfer Vectors for Mammalian Cells (Miller & Calos eds.); and “Current Protocols in Molecular Biology” (Ausubel, et al. eds., Wiley & Sons). Cell biology, protein chemistry, and antibody techniques can be found in “Current Protocols in Protein Science” (Colligan, et al. eds., Wiley & Sons); “Current Protocols in Cell Biology” (Bonifacino, et al., Wiley & Sons) and “Current Protocols in Immunology” (Colligan et al. eds., Wiley & Sons.). Reagents, cloning vectors, and kits for genetic manipulation referred to in this disclosure are available from commercial vendors such as BioRad, Stratagene, Invitrogen, ClonTech, and Sigma-Aldrich Co.

Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Other texts useful include Creating a High Performance Culture (Aroselli, Hu. Res. Dev. Pr. 1996) and Limits to Growth (D. H. Meadows et al., Universe Publ. 1974). Tissue culture supplies and reagents are available from commercial vendors such as Invitrogen, Nalgene-Nunc International, Sigma Chemical Co., Chemicon International, and ICN Biomedicals.

### EXAMPLE

1. Introduction

This example describes the development of efficient culture systems to maintain long-term growth of undifferentiated hESCs on a commercially available human feeder-layer as well as in feeder-free conditions in a defined serum-free medium that contains bFGF, insulin, and ascorbic acid.

Human ESCs, derived from the inner cell mass, have the capacity for long-term undifferentiated growth in culture, as well as the theoretical potential for differentiation into any cell type in the human body. These properties offer hESCs as a potential source for transplantation therapies and as a model system for studying mechanisms underlying mammalian development. Long-term cultivation of undifferentiated hESCs in a “biologics”-free—i.e., feeder-, serum-, and conditioned-medium-free—condition will be crucial for providing an unlimited supply of well-characterized healthy cells for cell-based therapies, as well as for directing the lineage-specific differentiation of hESCs.

To discover the minimal essential conditions needed to support the long-term growth of undifferentiated hESCs, morphological analysis was used to assess the developmental stage of hESCs at different times. For these analyses hESCs were grown in a 6- or 12-well plate to maturation (day 6 or 7 after seeding) before being fixed and visualized under a phase contrast microscope. Cellular immunofluorescence was also employed to assess the state of differentiation of hESCs. To perform these studies, hESCs were grown to maturation (day 6 or 7 after seeding) in a 12- or 24-well plate with a round cover slide in the bottom of each well. The cells were then fixed with 4% paraformaldehyde and blocked in PBS buffer containing 0.2% Triton X-100 and 2% BSA. Next, the cells were incubated with a primary antibody (Oct-4, SSEA-1, SSEA-3, SSEA-4, Tra-1-60, Tra-1-81, alkaline phosphatase, Myc, Map-2, Nkx2.5, bFGF (Santa Cruz Biotechnology, Inc.; Santa Cruz, Calif., world wide web: scbt.com) nestin, tyrosine hydroxylase (Chemicon International, Temecula, Calif., world wide web: chemicon.com), beta-tubulin (Sigma), p300, Tip6O, or acetylated H4 (K5, 8, 12, 16) (Upstate Biotechnology, Lake Placid, N.Y., world wide web: upstate.com) in wash buffer (0.1% Triton X-100 in PBS) at 4° C. overnight, and then with secondary antibody (Molecular Probe; Eugene, Oreg., world wide web: probes.com) in wash buffer at room temperature for 45 minutes. After further staining with DAPI, cells were mounted onto a microscope slide and visualized under an immunofluorescence and deconvolution microscope. The state of differentiation of hESC was further assessed by generating (via lentiviral-mediated transduction) hESCs carrying a reporter gene (enhanced green fluorescence protein (EGFP)) under control of the Oct-4 promoter. Using these transfected hESCs (carrying Oct-4 driven EGFP), the undifferentiated state of hESCs can be visualized by green fluorescence (indicating Oct-4 expression).

2. Cell Lines

The human NIH-approved ESC lines H1 and H9 were obtained from Wicell Research Institute (Madison, Wis., world wide web: wicell.org). Each cell line was originally maintained on mitomycin C-inactivated MEF (Specialty Media, Inc., Phillipsburg, N.J., world wide web: specialtymedia.com) in media consisting of 80% DMEMIF-12 or KO-DMEM, 20% Knockout Serum Replacement, 2 mM L-alanyl-L-glutamine (GlutaMax) or L-glutamine, 1× MEM nonessential amino acids, 100 μM β-mercaptoethanol (all from Invitrogen, Carlsbad, Calif., world wide web: invitrogen.com), and 4 ng/mL bFGF (PeproTech Inc., Rocky Hill, N.J., world wide web: peprotech.com). Cells were originally passaged once a week by treatment with dispase according to the instructions provided with the cell lines. Human ESCs on human feeder layers or on Matrigel-(Becton Dickinson, Bedford, Mass.; www.bdbioscience.com) coated plates (see method of coating below) were maintained in DMEM/F-12 or KO-DMEM (80%), knockout Serum Replacement (20%), L-alanyl-L-glutamine or L-glutamine (2 mM), MEM nonessential amino acids (1×), β-Mercaptoethanol (100 μM), bFGF (20 ng/ml), and insulin (4 μg/ml). Human recombinant insulin was from Sigma (St. Louis, Mo.; httip://www.sigma.com).

Initially, the hESC lines were maintained on growth-arrested MEFs. The undifferentiated hESCs formed tightly packed colonies with small compact cells of high nucleus-to-cytoplasm ratio. The hESC colonies then expanded by anchorage to surrounding feeder cells and by loosely attaching to the underlying tissue culture plate. Cells were initially passaged by treatment with dispase once—a week. However, dispase treatment did not efficiently separate hESCs from surrounding MEF cells, nor did the treatment effectively dissociate hESC colonies during passaging. In fact, additional mechanical dissection steps were required to detach and break hESC colonies down to smaller pieces. Trypsin treatment was not an acceptable alternative in those culture conditions, because treatment sufficient to dissociate the cells was lethal to the majority of undifferentiated hESCs on feeder layers; the rare hESC colonies that survived had an unacceptably higher rate of spontaneous differentiation than the parent colonies.

Because of the shortcomings of the dispase and trypsin methods, a non-enzymatic dissection process that produced more uniformly undifferentiated hESC colonies than the enzymatic methods was used. In this procedure, colonies estimated as having more than 80% morphologically undifferentiated cells were selected to be split. The selected hESC colonies were separated from the surrounding feeder cells, sliced into pieces, and detached from the tissue culture plate with a sterile plastic pipette tip. Then, the dissected hESC colony pieces were transferred to a fresh feeder layer and allowed to attach overnight. Culture medium was replaced every other day. The hESCs were passaged by this procedure every seven days at a split ratio of 1:8 to 1:4. This procedure not only was less time-consuming, but also resulted in higher plating efficiencies and more uniformly undifferentiated hESC colonies than the enzymatic methods. Although the use of one category of additives was eliminated, the problem of intimate contact with animal cells obviously persisted.

3. A Xeno-Free Serum-Free Feeder Layer

Next, in order to establish a culture system that was free of non-human animal products, the human foreskin fibroblast (HFF) cell line Hs27 was used as a feeder layer. The human foreskin fibroblast (HFF) cell line, Hs27 (ATCC; Manassas, Va.; www.atcc.orp) was expanded to create a master bank of frozen cells. The HFFs were plated in gelatin pre-coated 60 mm plates or 6-well plates at a density of 1.7×10⁴/cm² and inactivated by irradiating at 50 Gy using a ¹³⁷CS gamma-irradiator before being used as feeder cells. Undifferentiated hESCs, although originally maintained on MEFs, were transferred to plates of HFFs that had been mitotically inactivated by gamma irradiation. In the first attempts to transfer the hESCs to the human feeder layers, far more differentiated cells compared to those grown on MEFs were observed. When dealing with hESCs, the undifferentiated state was assessed by three criteria: (a) distinctive and defining stage-specific morphology and size; (b) the expression of immunomarkers associated with pluripotency; and (c) the absence of immunomarkers associated with lineage commitment. The hESC colonies maintained on HFFs displayed a more irregular morphology, more elliptical and less round than those grown on MEFs. Human ESC colonies co-cultured with HFFs were considerably smaller than those grown on MEFs, suggesting that some of the factors produced by MEFs that support undifferentiated hESC growth were missing or insufficient in the HFF culture system. Immunostaining for the undifferentiated hESC markers Oct-4, SSEA-4, Tra-1-60, and Tra-1-81 indicated that the hESC colonies on HFFs contained mixed patches of undifferentiated (<30%) and differentiated cells, often separated by distinct borders.

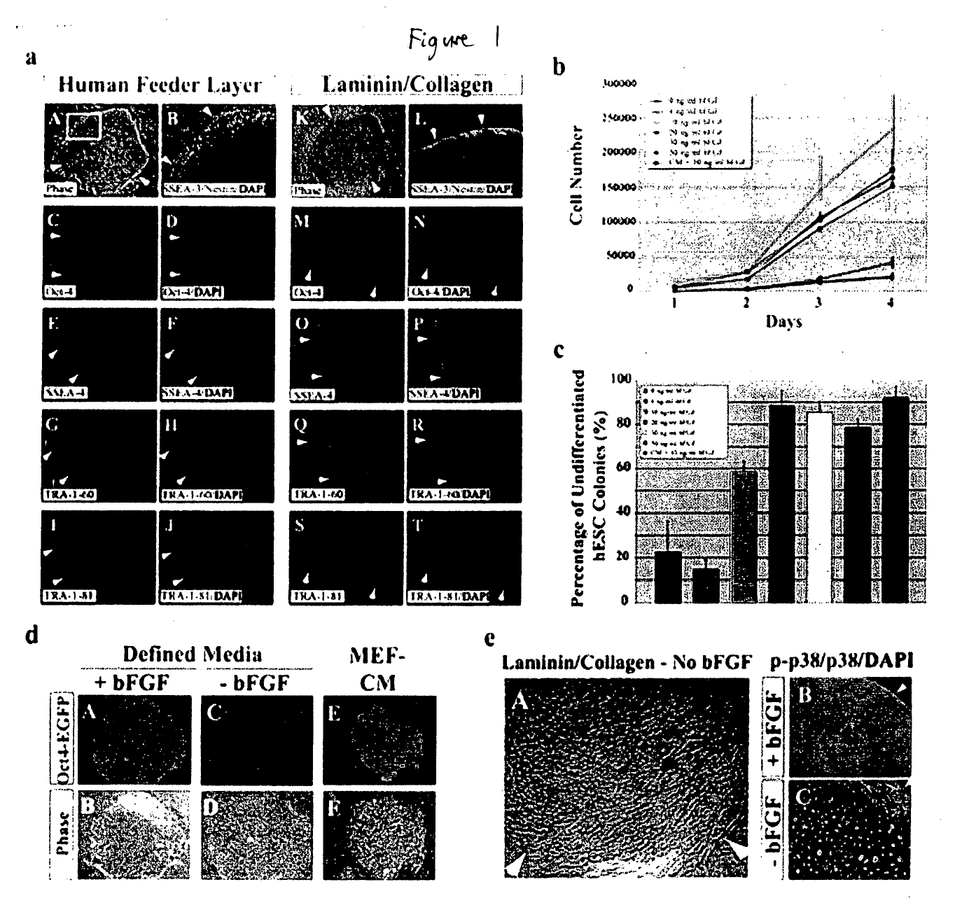

Surprisingly, it was discovered that, by increasing the bFGF concentration in the hESC medium to 20 ng/ml (from 4 ng/ml), the hESC colonies grown on the HFFs displayed the more round and undifferentiated morphological characteristics of hESC colonies grown on MEFs. These hESC colonies were also significantly larger, suggesting that bFGF promoted undifferentiated growth of hESCs on feeder layers. In addition to bFGF (20 ng/ml), the medium used to obtain these results contained DMEM/F-12 or KO-DMEM (80%), knockout Serum Replacement (20%), L-alanyl-L-glutamine or L-glutamine (2 mM), MEM nonessential amino acids (1×), and β-Mercaptoethanol (100 μM). In this media, less than 80% undifferentiated hESC colonies were observed on HFF feeders on every passage. Using this system, we undifferentiated hESCs on HFF feeder layers for over 12 months ( more than 50 passages) have been maintained, thereby exhibiting sustained long-term undifferentiated growth as assessed both by morphological and immunological criteria (FIG. 1a, A-J). Specifically, hESCs maintained on HFFs displayed uniform undifferentiated morphology (FIG. 1a, A) as well as high expression levels of Oct-4, SSEA-4, Tra-1-60, and Tra-1-81 (FIG. 1a, C-J), but not SSEA-1 (not shown). Only cells at the edge of the colonies exhibited—as expected—the classic signs of early differentiation: flat epithelial cell-like morphology; expression of the cell surface marker SSEA-3 and the neural/beta-cell precursor marker nestin (FIG. 1a, B). Cells that migrated beyond the edge of the colonies continued, as classically observed, to differentiate further into large elliptical cells that persisted in expressing nestin (suggestive of neuroectodermal commitment) and appropriately now downregulated SSEA-3 (FIG. 1a, B, red arrows).

4. Replacing Conditioned Media and Feeder Cells with Defined Components

A previous report indicated that MEF-conditioned media could support undifferentiated growth of hESCs on substrata such as laminin or laminin-collagen combinations (commercially known as Matrigel). The mechanism by which MEF-conditioned media exerts these effects is unknown, and it could involve the presence of growth factors, removal of toxic factors from the medium, or both. Based on the discovery that bFGF (at relatively high concentrations) promotes undifferentiated growth of hESCs on HFFs, it was decided to test whether bFGF promotes undifferentiated growth of hESCs on matrix proteins in the absence of feeder cells. Gelatin pre-coated plates were incubated with a commercially-available combination of laminin and collagen known as Matrigel (Growth factor reduced, Becton Dickinson) [diluted 1:30 in cold DMEM/F-12] at 4° C. overnight. The growth of hESCs on laminin/collagen-coated plates in the defined hESC media containing 20 ng/ml bFGF was examined. Over 80% of hESC colonies maintained on laminin/collagen-coated plates in each passage were highly compact and undifferentiated, as evidenced by their morphology and expression of Oct-4, SSEA-4, Tra-1-60, and Tra-1-81 by day 7 (FIG. 1a, K-T; 1c). The colonies on laminin/collagen had a more uniform morphology than those grown on HFFs, as indicated by the presence of an even narrower edge of SSEA-3-positive “transitional” (imminently-differentiating) cells (FIG. 1a, L, red) (compare to (FIG. 1a, B, red). Undifferentiated hESC colonies have been maintained for over 8 months (more than 32 passages) on laminin/collagen-coated plates, indicating that long-term undifferentiated growth of hESCs has been sustained.

To further assess the effect of bFGF on hESC undifferentiated growth, short-term proliferation assays of hESCs maintained under the feeder-free condition in the defined hESC media containing 0, 4, 10, 20, 30, or 50 ng/ml bFGF were performed. The growth rate (FIG. 1b) and the percentage of undifferentiated colonies (FIG. 1c) in response to bFGF doses were compared to those of hESCs maintained in MEF-conditioned media (MEF-CM) (the latter also actually “spiked” with an additional 10 ng/ml bFGF). In the defined media containing no bFGF or a low concentration of bFGF (4 ng/ml), hESCs displayed significantly slow growth (FIG. 1b) and high differentiation rates (FIG. 1c). In fact, in the absence of bFGF, approximately 80% of the hESC colonies maintained on laminin/collagen had a completely differentiated morphology (a typical differentiated colony is shown in FIG. 1e, A) and ceased expressing Oct-4 (not shown) upon their first passage, further suggesting that bFGF activity is essential for maintaining hESCs in an undifferentiated state. In media supplemented with bFGF at a concentration ranging from 10 to 50 ng/ml, hESCs displayed a growth rate comparable to that maintained in MEF-CM (FIG. 1b), while the optimal proportion of undifferentiated hESCs, comparable to MEF-CM, appeared to be maintained at 20 ng/ml bFGF (FIG. 1c). To further affirm the role of bFGF on hESC undifferentiated growth, hESCs carrying a reporter gene (enhanced green fluorescence protein [EGFP]) under control of Oct-4 promoter were generated (via lentiviral-mediated transduction). Using these transfected hESCs (carrying Oct-4 driven EGFP), it was observed that hESC colonies cultivated under the feeder-free condition displayed an undifferentiated morphology and strong green fluorescence (indicating Oct-4 expression) in the defined media containing 20 ng/ml bFGF, comparable to those maintained in MEF-CM, while more than about 70% of cells inside the colonies displayed a differentiated morphology and ceased Oct-4 expression in the absence of bFGF upon their first passage (day 7 after seeding) (FIG. 1d; also see FIG. 6a). Taken together, these results indicate that bFGF is a critical component in any defined hESC media for sustaining undifferentiated growth and, at the proper concentration, may substitute for feeder cells or MEF-conditioned media. To further support this conclusion, MEF-CM was examined for the presence of bFGF and it was found that endogenous bFGF (˜8-10 ng/ml) was, indeed, present in MEF-CM (see FIG. 6b), supporting that bFGF is, in fact, an essential factor in MEF-CM required for undifferentiated hESC growth.

To help understand the molecular mechanisms underlying bFGF's role in maintaining undifferentiated growth of hESCs, the mitogen-activated protein kinase (MAPK) signaling pathway was examined. However, no changes of phosphorylation levels of p38 MAPK were detected with increased bFGF concentrations by Western blot analysis, suggesting that p38 MAPK activation is not involved in bFGF-mediated hESC self-renewal. Next, using immunocytochemical analysis to better visualize individual cells, it was observed that an unphosphorylated inactive form of p38 MAPK was present robustly in undifferentiated hESCs maintained in the defined media (containing 20 ng/ml bFGF) (FIG. 1e, B). I In the absence of bFGF, however, the unphosphorylated form of p38 MAPK remained present in most of the large cells inside the differentiated hESC colony, some of the large differentiated cells (about 5%) displayed high levels of p38 phosphorylation (FIG. 1e, C, red), suggesting that p38 MAPK was activated and could be involved in differentiation of those cells. These observations suggested that bFGF is essential for maintaining hESCs in an undifferentiated state in part through deactivating p38 MAPK, and that p38 MAPK signaling activation might be involved in some aspects of hESC differentiation in the absence of bFGF.

5. The Fundamental Requirements for Sustained Undifferentiated Growth

Having determined that substantial numbers of undifferentiated hESCs could be maintained over long periods in feeder-free environments using the bFGF-supplemented media described above, the necessity of other components in the medium to maintain hESCs in an undifferentiated state was next examined. “Knockout Serum Replacement” contains insulin, transferrin, ascorbic acid, amino acids, and AlbuMAX (a chromatographically-purified lipid-rich bovine serum albumin [BSA] with low IgG content, but nevertheless a xeno-derived product). Accordingly, whether insulin, transferrin, BSA, and ascorbic acid were essential components was assessed, in combination with bFGF, for maintaining hESCs in an undifferentiated state. The serum replacement components insulin (20 μg/ml), transferrin (8 μg/ml), albumin (AlbuMAX )(10 mg/ml), and ascorbic acid (50 μg/ml) were added to a base medium that consisted of DMEM/F-12 or KO-DMEM with bFGF (20 ng/ml), L-alanyl-L-glutamine or L-glutamine (2mM), MEM essential amino acids solution (1×), MEM nonessential amino acids solution (1×), and β-mercaptoethanol (100 μM). To assay for the differentiation-forestalling activity of each of these components, undifferentiated hESCs were seeded on laminin/collagen-coated plates and cultivated for seven days in media containing one or more of the individual components. The degree of differentiation of the colonies was judged by defining morphology and Oct-4 expression. When all of the components were present, more than 70% of the HESC colonies had a highly compact undifferentiated morphology and expressed Oct-4 (FIG. 2a, A-C), suggesting that these factors were sufficient to support undifferentiated growth of a substantial number hESCs. In the absence of transferrin, fewer total hESC colonies were observed, but more than 70% of the hESC colonies that were present had a highly compact undifferentiated morphology and expressed Oct-4 (FIG. 2a, E-G). In the absence of albumin, hESC colonies were more flat and spread out, but more than 70% of the cells that were present nevertheless continued to express Oct-4 and exhibited a highly compact undifferentiated morphology (FIG. 2a, I-K). However, if ascorbic acid was omitted from the media, the colonies often became very dense at their core and necrotic (FIG. 2a, D,H,L, red arrows), suggesting that ascorbic acid was an essential component for maintaining the well-being as well as the undifferentiated growth of hESCs.

When either bFGF or insulin was omitted from the media, more than 90% of the colonies appeared to differentiate completely within the first passage, as indicated by their differentiated morphology (FIG. 2b, A,B,D,E), their complete loss of Oct-4 expression, and their expression of the cell surface marker SSEA-1 (FIG. 2c, B,C). Conversely, undifferentiated hESCs maintained in media containing both bFGF and insulin did not express SSEA-1 (FIG. 2c, A). Large round cells were typically present in media that contained only insulin (FIG. 2b, A, B) and elliptically-shaped cells were present in media that contained only bFGF (FIG. 2b, D, E), suggesting that insulin and bFGF might have distinct effects on hESC fate. The different effects of insulin and bFGF were accentuated further in media lacking ascorbic acid. In the absence of ascorbic acid and in media containing only insulin, the growth of differentiated hESCs was simply slower (FIG. 2b, C). In the absence of ascorbic acid and in media containing only bFGF, the appearance of cyst-like structures and necrotic cells within the dense cores of growing differentiated hESC colonies (FIG. 2b, F, red arrow) became more severe. Taken together, these results indicated that, in addition to bFGF, insulin and ascorbic acid were also essential—perhaps in a collaborative manner—for maintaining substantial numbers of hESCs in a healthy undifferentiated state. Although albumin and transferrin are not crucial components for sustaining the undifferentiated growth of hESCs, they might abet survival or maintenance of a normal colony shape.

Interestingly, bFGF has been reported to regulate cell proliferation and differentiation by inducing chromatin remodeling. Therefore, to further study the molecular mechanism underlying the maintenance by bFGF and insulin of pluripotency in hESCs, the epigenetic chromatin states of hESCs in response to these components was examined. In the presence of both bFGF and insulin, undifferentiated hESCs are associated with acetylated histone H3 and H4, and strong expression of Myc and histone acetyltransferase (HAT) p300 and Tip60 (FIG. 2c, D-F, I-K). However, when either bFGF or insulin was omitted from the media, the differentiated cells showed undetectable or weak immunoreactivity to acetylated H3 and H4, Myc, Tip60 HAT, and nuclear focal localization of p300 HAT (FIG. 2c, G, H, L, M). The transcriptional activator Myc has been shown to recruit HAT complexes, such as Tip60 complex, to induce histone acetylation. In general, acetylated histones correlate with a transcriptionally active (“open”) chromatin state, whereas deacetylated histones correlate with a transcriptionally repressed (“closed”) chromatin state. Without wishing to be bound to a particular theory, the results above suggest that the presence of both bFGF and insulin is essential for maintenance of an acetylated transcriptionally-active chromatin state in undifferentiated hESCs, while the absence of either bFGF or insulin induces differentiation that results in the formation of a hypo-acetylated, repressed chromatin structure.

Further, whether other growth factors could support undifferentiated growth of hESCs in a manner comparable to bFGF was also examined. For example, the effects of acidic fibroblast growth factor (aFGF), epidermal growth factor (EGF), insulin-like growth factor-I (IGF-I), insulin-like growth factor-II (IGF-II), platelet-derived growth factor-AB (PDGF), vascular endothelial cell growth factor (VEGF), activin-A, and bone morphogenic protein 2 (BMP-2) on the growth of hESCs was studied. All the growth factors were dissolved in a PBS buffer that contained 0.5 % BSA, 1 mM DTT (Dithiothreitol) and 10% glyceral as a 10 μg/ml (500×) stock solution and stored in aliquots at −80° C. These factors were added individually to the hESC cultures at a concentration of 20 ng/ml, in the absence of bFGF or in the presence of a low concentration of bFGF (4 ng/ml). Seven days after seeding undifferentiated hESCs on laminin/collagen-coated plates, the cultures were examined. In every case, most colonies (greater than 70%) consisted of dense centers containing cyst-like structures and necrotic cells (FIG. 2d, A-D, red arrows0 surrounded by a flat layer of fibroblast-like cells. Although colony morphologies differed slightly depending on the growth factor used (representative colonies are shown in (FIG. 2d, A-D]), none of the factors was sufficient for replace bFGF in maintaining undifferentiated growth of hESCs. Interestingly, although most cells became differentiated when using these alternative growth factors, a minority of the small colonies ( fewer than 30%) retained compact morphologies (blue arrows, FIG. 2d, E) and continued to express Oct-4 (FIG. 2d, F, G).

6. Providing a Minimal Defined Matrix Yields a “Self-Supporting” System

Having established that bFGF, insulin, and ascorbic acid were important minimal components of a feeder-free culture system, the growth of hESCs on purified matrix proteins, including human laminin-, fibronectin-, or collagen IV-coated plates in hESC media containing 20 ng/ml bFGF, was further examined. Similar to hESCs maintained on laminin/collagen-coated plates, more than 80% of the hESC colonies remained undifferentiated on surfaces coated with laminin alone, as indicated by their classic undifferentiated morphology (FIG. 2e, A) and their expression of Oct-4 (FIG. 2e, B, C), suggesting that the laminin portion of Matrigel is the critical component. In contrast, the majority of the hESC colonies (more than 70%) maintained on human fibronectin (FIG. 2e, D), human collagen IV—(FIG. 2e, E), or, as a control, gelatin-coated plates (FIG. 2e, F), displayed a more differentiated morphology upon the first passage, leaving only a minority (less than 30%) of small colonies bearing a compact, undifferentiated morphology. Interestingly, the colonies of undifferentiated cells maintained under the feeder-free conditions (on either laminin or laminin/collagen-coated plates) appeared to be associated with a monolayer of hESC—derived fibroblastic cells (FIG. 1a, K, red arrows; FIG. 2e, A; FIG. 3a, A, E ) that expressed nestin (e.g., FIG. 1a, L, red arrows) and vimentin (e.g., FIG. 3b, K, L). This observation suggested that these cells may spontaneously act as “auto feeder layers” for the very same undifferentiated hESC colonies from which they were derived, preventing them from differentiating, rendering the system “self-contained”, “self-supporting”, and precluding the need for exogenous “biologics”—including human-derived components, as discussed below.

7. Self-Renewal and Pluripotency in a “Self-Contained,” Defined Biologics-Free System

In the course of successfully affirming the self-renewing capacity of these hESCs, another interesting observation emerged, reinforcing the ability to provide completely characterized components for growth of the cells. To demonstrate the self-renewal of undifferentiated hESCs maintained under the above-described defined biologics-free culture conditions, hESCs were treated with trypsin, dissociated into a single cell suspension, and then cultivated under the defined conditions (FIG. 3a). Undifferentiated mature-sized single-cell-derived hESC colonies began to appear after 4-7 days in vitro (FIG. 3a, C-F). A 12.6±3.8% cloning efficiency of hESCs cultured under the defined conditions was observed. This observation contrasted starkly with the extremely poor cloning efficiency that has been reported previously using culture conditions employing feeder cells or conditioned media. In fact, complete cell death has been observed when single undifferentiated cells dissociated by trypsin treatment were passaged onto exogenous feeder cells or in conditioned media (particularly for hESCs that have never been exposed to trypsin digestion, e.g., HES-25). However, undifferentiated hESCs displayed unexpectedly high passaging efficiency with trypsin treatment under the defined biologics-free culture conditions. One explanation is that the dissociated single cells seeded highly efficiently on a substrate containing laminin in the defined hESC media. In addition, the colonies of undifferentiated cells appeared to be associated with a monolayer of hESC—derived fibroblastic cells (FIG. 3a, C-D) that expressed vimentin (FIG. 3b, K, L). These differentiated cells may spontaneously act as “auto feeder layers” for the very same undifferentiated hESC colonies from which they were derived, preventing them from differentiating. Stated another way, the system appeared to become “self-contained” or “self-supporting” by exploiting the fact that, by definition, pluripotent hESCs will inevitably include, among its many products of differentiation, those lineages that have heretofore been supplied exogenously as “foreign” human feeder cells. The system now allowed these hESCs to produce their own support (“feeder”) cells. To date, undifferentiated hESC colonies have been passaged with trypsin treatment for more than 30 passages under the defined culture conditions, as evidenced by their uniform undifferentiated morphology (FIG. 3a, F) as well as high expression levels of alkaline phosphatase, Oct-4, SSEA-4, Tra-1-60, and Tra-1-81 (FIG. 3b, A-J) . In addition, it was observed that hESCs passaged by either mechanical dissection or trypsin treatment maintained a stable karyotype (0/20 abnormal spreads) after a prolonged period of culturing under the defined conditions, while hESCs cultured under exogenous feeder or in conditioned media displayed a relatively frequent abnormality (2-4/20 abnormal spreads) when passaged by trypsin treatment.

As indicated above, to further affirm that undifferentiated hESCs are capable of self-renewal under these defined conditions, a reporter gene (EGFP) under control of the Oct-4 promoter was introduced via lentiviral-mediated transduction into subclones of undifferentiated hESCs. Infected cells, which incorporated only a single transgene (hence delineating clones), were cultivated under the feeder-free condition in the defined media containing 20 ng/ml bFGF for a prolonged period. A green (Oct-4 expressing) undifferentiated hESC colony subcloned from the infected cells is shown in FIG. 3c.

To affirm their continued pluripotency, undifferentiated hESCs after prolonged propagation under the above-described defined biologics-free conditions were injected intramuscularly into SCID mice. Teratomas developed with great efficiency in these mice. Histological analysis of teratomas generated in SCID mice revealed the presence of tissues of all three embryonic germ layers, including pigmented neural tissue (ectoderm); gut epithelium (endoderm); and adipose cells, blood vessels, cartilage, smooth muscle, and connective tissue (mesoderm) (FIG. 4a).

8. Efficient Lineage Specification Under the Defined Biologics-Free Conditions

The ability to maintain undifferentiated hESCs under an entirely defined biologics-free condition (e.g., serum-, feeder-, conditioned medium-free) not only facilitate clinical translation, but also make it possible to identify and control the true (i.e., minimal essential) conditions necessary to guide pluripotent stem cells towards a lineage-specific fate. Under the above-described biologics-free conditions, pluripotent hESCs have been efficiently directed towards at least two prototypical representative specific somatic lineages that hold therapeutic potential: differentiation toward cardiac tissue and differentiation toward neuronal tissue.

To direct cardiac differentiation, undifferentiated hESCs cultured under the defined biologics-free condition were detached and allowed to form embryoid bodies (EBs) in a suspension culture in a standard differentiation media consisting of KO-DMEM (80%), defined FBS (Hyclone) (20%), L-glutamine (2 mM), MEM nonessential amino acids (1×), β-Mercaptoethanol (100 μM). After permitting the EBs to attach to a tissue culture substrate, beating cardiomyocytes were observed in about one week, increased in numbers with time, and retained their contractility for over two months. These beating cells 9FIG. 4b, A) expressed markers characteristic of cardiomyocytes, such as cardiac transcription factors Nkx2.5 9FIG. 4b, B), MEF-2, and GATA-4, as well as cardiac myosin heavy chain (MHC) (FIG. 4b, C0.

Retinoic acid (RA) increases (though is not required for) neuronal differentiation of hESCs maintained on MEF-feeder cells if added to their differentiated EBs. In contrast, for undifferentiated hESCs maintained under these defined conditions, RA was sufficient to induce a complete sequence of neural differentiation (as indicated by progressive changes in morphology and expression of stage-specific markers) starting as early as the pluripotent undifferentiated stage rather than at the later EB stage. Upon exposure of undifferentiated hESCs to RA (10 μM), large differentiated cells within the core of the colony began to emerge (FIG. 5a, A, B) that ceased expressing Oct-4 9FIG. 5a, C) and began expressing the early differentiated stage marker SSEA-1 (FIG. 5a, D). These large differentiated cells continued to multiply and the colonies increased in size. These differentiating hESCs then formed floating clusters of cells (cytospheres) when transferred to a suspension culture in a defined serum-free media containing DMEM/F-12 (80%), knockout Serum Replacement (20%), L-alanyl-L-glutamine (2 mM), MEM nonessential amino acids (1×), and β-Mercaptoethanol (100 μM) for 4 days. FIG. 5b, A0. In the absence of bFGF and after permitting the cytospheres to attach to a tissue culture substrate, pigmented cells (typical of those in the central nervous system) (FIG. 5b, B,D) and β-III-tubulin- and MAP-2-expressing, exuberantly neurite-bearing cells (suggestive of neurons) (FIG. 5b, B, C; 5c) began to appear after about a week of cultivation, increased in numbers with time, and could be sustained for more than 3 months in a defined medium containing DMEM/F-12, N-2 supplement (1%), heparin (82 g/ml; micrograms per milliliter), VEGF (20 ng/ml; nanograms per milliliter), neurotrophin-3 (NT-3, 10 ng/ml), and brain-derived neurotrophic factor (BDNF, 10 ng/ml) had been added. A large proportion of these hESC -derived neuronal cells began to express tyrosine hydroxylase (FIG. 5d), suggesting a catecholaminergic or dopaminergic potential.

9. Summary and Conclusions

This example identifies the minimal essential components necessary to maintain primate embryonic stem cells, in particular hESCs, in a healthy, undifferentiated state capable of both prolonged propagation and then directed differentiation. Having discerned these molecular requirements, it became possible to derive conditions that would permit the substitution of poorly-characterized and unspecified biological additives and substrates (including those derived from animals) with entirely defined constituents. In other words, a defined serum-free, conditioned medium-free medium for the long-term cultivation of undifferentiated hESCs on not only human feeder layers but also under feeder-free conditions has now been invented. The studies described herein have led to the identification of bFGF, insulin, ascorbic acid, and laminin as the essential components of a minimal culture system that maintains hESCs in a healthy self-renewing pluripotent state (partially by the maintenance of an acetylated transcriptionally-active chromatin state). All are chemically defined components, enabling a “biologics”-free formulation. This defined culture system has the advantage of allowing hESCs to be expanded efficiently and stably following trypsin-mediated dissociation, not possible under previously-described culture systems containing feeder cells or conditioned media. Furthermore, to keep the system free from the need for any “foreign” biological additives, the fact that, by definition, pluripotent hESCs will inevitably include, among its many products of differentiation, those lineages that have heretofore been supplied exogenously as “foreign” human feeder cells has been exploited, and the system optimized to allow these hESCs to spontaneously produce their own support (“feeder”) cells. Therefore, this study provides a viable approach for providing a large supply of well-characterized, clinically-acceptable, healthy cells for cell-based therapies. In addition, having established individual components required for the undifferentiated growth of hESCs, it is now possible to assess more accurately the effects of other growth factors and compounds on the developmental fate of hESCs. As will be appreciated, defined media are crucial for directing a requisite number of pluripotent hESCs efficiently, uniformly, stably, and reproducibly towards a specific lineage for therapeutic purposes.

All patents and patent applications, publications, scientific articles, and other referenced materials mentioned in this specification are indicative of the levels of skill of those skilled in the art to which the invention pertains, and each of which is hereby incorporated by reference to the same extent as if each reference had been incorporated by reference in its entirety individually. Applicants reserve the right to physically incorporate into this specification any and all materials and information from any such patents and patent applications, publications, scientific articles, electronically available information, and other referenced materials or documents.

The specific media compositions, culture systems, and methods described in this specification are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications can be made to the invention disclosed herein without departing from the scope and spirit of the invention. The invention illustratively described herein suitably may be practiced in the absence of any element or elements, or limitation or limitations, which is not specifically disclosed herein as essential. Also, the terms “comprising”, “including”, “containing”, etc. are to be read expansively and without limitation. It must be noted that as used herein and in the appended claims, the singular forms “a”, “an”, and “the” include plural reference, and that the use of the word “or,” for example, as in the case of “a or b” may refer to a alone, to b alone, or to a and b together, unless the context clearly dictates otherwise.

The terms and expressions that have been employed are used as terms of description and not of limitation, and there is no intent in the use of such terms and expressions to exclude any now-existing or later-developed equivalent of the features shown and described or portions thereof, but it is recognized that various modifications are possible within the scope of the invention as claimed. Thus, it will be understood that although the present invention has been specifically disclosed by preferred embodiments and optional features, modification and/or variation of the disclosed elements may be resorted to by those skilled in the art, and that such modifications and variations are within the scope of the invention as claimed.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. Thus, it is understood that any dependent claim among the appended claims merely represents particular embodiments within the scope of the subject matter bounded by the claim(s) from which the claim depends, and the inventors reserve the right to pursue subject matter that is within the scope of a more broad claim but is not specifically recited in an appended claim.

1. A defined, isotonic culture medium that is essentially feeder-free and serum-free, comprising:
a. a basal medium;
b. an amount of bFGF sufficient to support growth of substantially undifferentiated mammalian stem cells;
c. an amount of insulin sufficient to support growth of substantially undifferentiated mammalian stem cells; and
d. an amount of ascorbic acid sufficient to support growth of substantially undifferentiated mammalian stem cells.

2. A culture medium according to claim 1 wherein the mammalian stem cells are primate stem cells.

3. A culture medium according to claim 2 wherein the primate stem cells are primate primordial stem cells.

4. A culture medium according to claim 3 wherein the primate primordial stem cells are human primordial stem cells.

5. A culture medium according to claim 4 wherein the human primordial stem cells are human embryonic stem cells.

6. A culture medium according to claim 1 wherein the substantially undifferentiated mammalian stem cells comprise cells that present at least one marker selected from the group consisting of alkaline phosphatase, Oct-4, SSEA-4, Tra-1-60, Tra-1-81, SSEA-1, SSEA-3, Myc, nestin, musashi, vimentin, acetylated histones, p300, Tip60, histone acetyltransferases, and histone deacetylases.

7. A culture medium according to claim 1 wherein the basal medium comprises DMEM, DMEM/F-12, or KO-DMEM, that contains essential amino acids and a carbon source that can be metabolized by the mammalian stem cells.

8. A culture medium according to claim 7 that has a low endotoxin level.

9. A culture medium according to claim 9 wherein the medium further comprises at least one of the following chemicals selected from the group consisting of non-essential amino acids, anti-oxidants, reducing agents, vitamins, organic compounds, inorganic salts, sodium pyruvate, transferrins, and albumins.

10. A culture medium according to claim 9 wherein the reducing agent is β-mercaptoethanol.

11. A culture medium according to claim 1 wherein the amount of bFGF ranges from about 1 ng/mL to about 20 μg/mL.

12. A culture medium according to claim 1 wherein the amount of bFGF is about 20 ng/mL.

13. A culture medium according to claim 1 wherein the amount of insulin ranges from about 1 ng/mL to about 20 mg/mL.

14. A culture medium according to claim 1 wherein the amount of insulin is about 20 μg/mL.

15. A culture medium according to claim 1 wherein the amount of ascorbic acid ranges from about 1 ng/mL to about 50 mg/mL.

16. A culture medium according to claim 1 wherein the amount of ascorbic acid is about 50 μg/mL (microgram/ml).

17. A system for culturing mammalian primordial stem cells in a substantially undifferentiated state, comprising:
a. a defined, isotonic culture medium according to claim 1; and
b. cell culture vessel includes a substrate comprising a matrix.

18. A system according to claim 17 wherein the mammalian primordial stem cells are primate primordial stem cells.

19. A system according to claim 18 wherein the primate primordial stem cells are human primordial stem cells.

20. A system according to claim 19 wherein the human primordial stem cells are human embryonic stem cells.

21. A system according to claim 18 wherein the matrix is an extracellular matrix.

22. A system according to claim 21 wherein the extracellular matrix is a cell-free matrix prepared from one or more matrix components.

23. A system according to claim 22 wherein the extracellular matrix comprises at least one molecule selected from the group consisting of laminin, fibronectin, collagen, and gelatin.

24. A system according to claim 18 wherein the matrix is provided by a primate feeder cell layer.

25. A system according to claim 24 wherein the primate feeder cell layer is a human feeder cell layer.

26. A system according to claim 25 wherein the human feeder cell layer comprises cells selected from the group consisting of human fibroblast cells, human stromal cells, and cells differentiated from human primordial stem cells.

27. A system according to claim 18 that comprises a plurality of culture vessels for passaging the primate stem cells from one culture vessel to another for continued culturing in a substantially undifferentiated state, wherein a culture vessel used in a subsequent passage comprises the same species of substrate as was used in the culture vessel from which the cells are being passaged.

27. A method of culturing mammalian primordial stem cells in a substantially undifferentiated state, comprising culturing the cells in a culture environment that is essentially free of xenogeneic feeder cells, added conditioned medium from feeder cells, and serum and which comprises a defined, isotonic culture medium according to claim 1.

28. A method according to claim 27 wherein the mammalian primordial stem cells are primate primordial stem cells.

29. A method according to claim 28 wherein said a culture environment comprises at least one component selected from the group consisting bFGF, insulin, ascorbic acid, laminin, or derivatives of such components in an amount sufficient to support substantially undifferentiated growth of primate primordial stem cells.

30. A method according to claim 28 wherein the primate primordial stem cells are human primordial stem cells.

31. A method according to claim 30 wherein the human primordial stem cells are human embryonic stem cells.

32. A method according to claim 30 wherein the primate primordial stem cells are isolated from blastocysts or 1-8 cell stage embryos.

33. A method according to claim 31 wherein the isolation is performed by morphology assessment and selecting as primate primordial stem cells, those cells which present at least one marker selected from the group consisting of Oct-4, SSEA-4, Tra-1-60, Tra-1-81, alkaline phosphatase, SSEA-1, SSEA-3, Sox-2, Myc, acetylated histones, p300, Tip6O, histone acetyltransferases (HATs), and histone deacetylases (HDACs).

34. An isolated cell population comprising substantially undifferentiated mammalian primordial stem cells cultured in accordance with the method of claim 27.

35. An isolated cell population according to claim 34 wherein the mammalian primordial stem cells are primate primordial stem cells.

36. An isolated cell population according to claim 35 wherein the primate primordial stem cells are human embryonic stem cells.

37. An isolated cell population according to claim 36 that is a cell line comprised of human embryonic stem cells.

38. A cell line according to claim 37 comprising normal human embryonic stem cells.

39. A cell line according to claim 37 comprising abnormal human embryonic stem cells.

40. A method of identifying a factor that promotes differentiation or helps maintainan undifferentiated state of mammalian primordial stem cells, comprising:
a. exposing a test compound to substantially undifferentiated primate primordial stem cells being cultured in a growth environment that is essentially free of xenogeneic feeder cells, added conditioned medium from feeder cells, and serum and which comprises a defined, isotonic culture medium according to claim 1;
b. determining whether the substantially undifferentiated primate primordial stem cells begin to differentiate or better sustain the undifferentiated state following exposure to the test compound; and, if so,
c. identifying the test compound as a factor that promotes differentiation or help maintaining undifferentiated state of substantially undifferentiated primate primordial stem cells.

41. A method according to claim 38,wherein the mammalian primordial stem cells are primate primordial stem cells.

42. A method according to claim 41, wherein said identified factor promotes cardiac differentiation.

43. A method according to claim 41, wherein said identified factor promotes neuronal differentiation.

44. A method according to claim 43 wherein said identified factor comprises retinoic acid (RA) that promotes lineage-specific differentiation into neural, neuronal, and pigmented-cells.

45. A method according to claim 44 wherein lineage-specific differentiation comprises differentiation into cells which present at least one marker selected from the group consisting of Map-2, β-III-Tubulin, and tyrosine hydroxylase.

46. A method according to claim 40 comprising a high throughput method.

47. A method of cell therapy, comprising administering to a patient in need of such therapy an isolated cell population according to claim 34, wherein the cells are substantially undifferentiated human embryonic stem cells or differentiated cells derived from substantially undifferentiated human embryonic stem cells.

48. A method according to claim 47 wherein the patient suffers from a disorder selected from the group consisting of heart diseases, diabetes, liver diseases, disorders caused by a single gene defect, neurodegenerative diseases, tumors, cancers, strokes, spinal cord injury or diseases, Alzheimer's diseases, Parkinson's diseases, multiple sclerosis, and amyotrophic lateral sclerosis (ALS).
